(19) United States
(12) Patent Application Publication
(10) Pub. No.: US 2026/0117309 A1
(43) Pub. Date: Apr. 30, 2026

(54) Detection, Quantification, and Validation of microRNA Biomarkers

(71) Applicant: Anastassia Kanavarioti, El Dorado Hills, CA (US)

(72) Inventor: Anastassia Kanavarioti, El Dorado Hills, CA (US)

(21) Appl. No.: 19/199,818

(22) Filed: May 6, 2025

Related U.S. Application Data
(60) Provisional application No. 63/643,741, filed on May 7, 2024.

(51) Int. Cl. C12Q 1/6886

(52) U.S. Cl. CPC C12Q 1/6886; C12Q 2600/156; C12Q 2600/178

(57) ABSTRACT

An amplification-free assay, a detector suitable for nucleic acid trace measurements, and a protocol designed to deliver miRNA copies at an approximate +/−20% accuracy across all concentrations. This unprecedented accuracy led to the conclusion that miRNA copy numbers must be normalized to the same RNA content which in turn illustrated (i) independence from age, gender, and ethnicity as well as (ii) equivalency between serum and urine. The technology is well-positioned to overcome the inconsistencies in the miRNA field and revolutionize the validation of miRNA biomarkers.

### CROSS-REFERENCE TO RELATED APPLICATIONS

This application claims the benefit of U.S. provisional application No. 63/643,741, filed on May 7, 2024, which is incorporated herein in its entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Dec. 23, 2025, is named 68812-245327_SL.xml and is 30,484 bytes in size.

### BACKGROUND

The incidence of cancer has not declined despite worldwide prevention efforts [Siegel et al. (2023); WHO, Cancer]. The cancer incidence rate climbs steadily with age and reaches 1 for every 100 people 60 years old and older [NIH/NCI]. Most adults receive an annual or biannual physical medical examination, which does not include a multicancer check. There are specialized tests for some prevalent cancer indications, but they are invasive, often painful, and can lead to unacceptable false positive or false negative results [Komen.org; WHN Mammograms; NHSE, Prostate; Borbiev et al. (2023); Kane et al. (2022); Stocik et al. (2023)]. Rigorous observational studies in Europe have failed to find an effect of mammography screening. Mammography screening results in patients with breast cancer from among healthy women and increases the number of mastectomies performed [Komen.org; WHN Mammograms]. A blood test for a prostate-specific antigen (PSA) measures the level of PSA, which is a substance made by the prostate. The levels of PSA in the blood might be greater in men who have prostate cancer, but the PSA test exhibits a 15% false positive rate that leads to unnecessary surgical biopsies [NHSE, Prostate; Borbiev et al. (2023)]. There is no single diagnostic test for pancreatic cancer. Serum levels of the antigen CA 19-9 higher than 37 U/mL are exploited for the diagnosis of pancreatic cancer in symptomatic patients, but they are not useful as screening markers in asymptomatic individuals because of their low positive predictive value [Kane et al. (2022); Stocik et al. (2023)]. Definitive diagnosis requires a series of imaging scans, blood tests and biopsies, and those tests are typically performed after symptoms appear. Pancreatic cancer is called a “silent” disease because it may cause patients to experience no symptoms until it is too late. Early detection is promising for curing cancer and saving lives. Early detection relies heavily on minimally invasive tests using a blood sample, urine, saliva, or other biological fluids, so-called liquid biopsies [Connal et al. (2023); Armakolas et al. (2023)]. Multiple cancer blood tests using known protein cancer biomarkers [Cohen et al. (2018)] or circulating tumor DNA shed from tumors [Beer (2020); Duffy & Crown (2023)], as well as DNA methylation profiles [Nickolson et al. (2023)], are currently being tested in large trials to replace exploratory invasive tissue biopsies and support clinical decisions in symptomatic individuals.

A 2001 seminal publication by Victor Ambros summarized the findings regarding the function of miRNAs [Ambros (2001)], a class of small noncoding RNAs with a length of 18 to 25 nucleotides [Bartel (2004); Kozomara et al. (2019)]. Ambros proposed them to be the “tiny regulators” that control posttranscriptional gene expression, including that related to cell growth, differentiation, development, and apoptosis. miRNAs are abundant in most eukaryotes, and approximately 2500 known miRNAs are common to all humans [Kozomara et al. (2019); Alles et al. (2019)]. In 2008, Mitchell and coworkers illustrated that miRNAs are stable in blood, which renders them viable biomarkers [Mitchell et al. (2008)] and empowered more than 75,000 peer-reviewed studies across the world [Valihrach et al. (2020); Smolarz et al. (2022); Porzycki et al. (2018); Zografos et al. (2019); Jenike & Halushka (2021); Filella & Foj (2017); Sequeira et al. (2023); Gahlawat et al. (2023); Gao et al. (2016); Alahdal et al. (2023)]. Seven hundred studies each investigated the expression of miR-375 and miR-141 for most cancer indications. Multiple studies have confirmed that these miRNAs are overexpressed in the serum of cancer patients compared to healthy controls [Mitchell et al. (2008); Porzycki et al. (2018); Filella & Foj (2017)]. A recent review listed 29 medical studies with a total of approximately 7,000 subjects in which elevated miR-21 levels were reported across neoplastic and nonneoplastic diseases [Jenike & Halushka (2021)]. miR-21 may not be a biomarker for a specific disease, but it is a multi-cancer, multi-disease biomarker. One should be able to determine from a regular medical exam whether his or her miR-21 level is markedly higher than the level found in healthy subjects (HL). Besides miRNAs, no other biomarker has been explored so intensely, and found to be involved in cancer onset, progression, metastasis, or survival. Often, the miRNA data from cancer patients overlap with the data from healthy controls, and it is only the median from cancer samples which may differ from the median of the healthy samples [Sequeira et al. (2023); Godoy et al. (2019); Hindson et al. (2013); Jet et al. (2021); Mestdagh et al. (2014); Seyhan (2023)]. For example, droplet digital PCR (ddPCR) data from patients with urological cancers reported copy numbers per μL of plasma for miR-126, miR-141, miR-155, miR-182 and miR-375 in the range of 0 to 3,000, 0.5 to 4, 2 to 40, 0 to 20 and 0 to 40, respectively [Sequeira et al. (2023)]. Notably, a similar variation was seen in the cancer samples and in the healthy samples. This type of data spread is not atypical for miRNA quantification and has prevented the validation of selected miRNAs as biomarkers in cancer diagnostic assays.

The overlap of miRNA data between diseased and healthy subjects, quantitative disagreement among studies, and the determination of whether a certain miRNA acts as an oncogene or as a tumor suppressor have been attributed to differences in biospecimen collection methods, study protocols, choice of reference, analytical methods, population variation, disease stage, etc. [Godoy et al. (2019); Hindson et al. (2013); Jet et al. (2021); Mestdagh et al. (2014); Seyhan (2023)]. To improve the statistics, the collective response from a miRNA panel has been widely proposed. To the best of our knowledge no miRNA study has reported a zero data overlap between healthy samples and samples with a certain disease condition, i.e., 100% sensitivity and 100% specificity yet. The concentration of miRNAs in blood is in the low femtomolar (fM) range, which is a billion-fold less than the micromolar (μM) range which is required by typical UV-Vis analytical tools. Current methods for profiling the relative abundance of miRNAs in biological fluids or tissues include small RNA sequencing, reverse transcription-quantitative PCR (RT-qPCR), droplet digital PCR (ddPCR), and microarray hybridization [Sequeira et al. (2023); Godoy et al. (2019); Hindson et al. (2013); Jet et al. (2021); Mestdagh et al. (2014); Seyhan (2023)]. While identification works well with these tools, the quantification accuracy and choice of reference have been questioned and may be partially responsible for the conflicting conclusions [Godoy et al. (2019); Hindson et al. (2013); Jet et al. (2021); Mestdagh et al. (2014); Seyhan (2023)].

The diverging literature reports illuminate the need for a novel analytical tool of exceptional accuracy. Asymptomatic individuals, especially those aged >60 years with or without a family cancer history, worry about having cancer. A liquid biopsy test to label an asymptomatic individual cancer-free will ease worrying and reduce unnecessary doctor visits. There remains a need for such a test that will be a valuable addition to the regular physical medical examination.

### SUMMARY

Provided for herein is a method of validating a miRNA as a biomarker indicative of a disease. In certain embodiments, the method comprises (a) measuring a control miRNA copy number (H1) in a sample of isolated total RNA from a biospecimen of a control subject without the disease (isolated total RNA control sample) using an osmylated-probe that targets the miRNA and a nanopore detection method, wherein the accuracy of the control miRNA copy number measurement is protocol-defined; (b) repeating step (a) for one or more additional control samples (H2 to Hn); (c) normalizing the miRNA copy numbers of the additional control samples H2 to Hn to the RNA content of the isolated total RNA control sample and selecting an average miRNA copy number from H1 to Hn as a control miRNA copy number (Hc); (d) measuring a disease miRNA copy number (D1), of the same miRNA measured in (a), in the isolated total RNA from a biospecimen of a disease subject confirmed to have the disease using the same osmylated probe used in (a) that targets the miRNA and the nanopore detection method, wherein the accuracy of the disease miRNA copy number measurement is protocol-defined; (e) repeating step (d) for one or more additional disease samples (D2 to Dn); (f) for overexpression, normalizing the miRNA copy numbers D1 to Dn of the disease samples to the RNA content of the isolated total RNA control sample and then dividing the disease sample normalized miRNA copy number values (D1(norm) to Dn(norm)) by the miRNA copy number of the control sample (Hc) to obtain a series of ratios of the normalized disease sample miRNA copy number to the control sample miRNA copy number (D1(norm)/Hc to Dn(norm)/Hc) or for underexpression, normalizing the miRNA copy numbers D1 to Dn of the disease samples to the RNA content of the isolated total RNA control sample and then dividing the miRNA copy number of the control sample (Hc) by the disease sample normalized miRNA copy number values (D1(norm) to Dn(norm)) to obtain a series of ratios of the normalized disease sample miRNA copy number to the control sample miRNA copy number (Hc/D1(norm) to Hc/Dn(norm)); and (g) making a determination whether the miRNA is a validated biomarker based on the ratios determined in (f).

Also provided for herein is a method of detecting a disease in a subject using as a biomarker a miRNA validated for the disease according to a validation method of this disclosure.

Also provided for herein is a method of detecting a disease in a subject: (i) wherein the method comprises incubating a predetermined threshold amount of an osmylated-probe that binds to a miRNA biomarker of the disease with a sample of a specified amount of RNA isolated from the subject, and using a nanopore detection method to determine whether there is an excess of probe in the incubated sample (detection) or an excess of miRNA biomarker in the incubated sample (silencing), wherein silencing is indicative of an elevated amount of the miRNA biomarker and that the subject has the disease or (ii) wherein the method comprises incubating a predetermined threshold amount of an osmylated-probe that binds to a miRNA biomarker of the disease with a sample of a specified amount of RNA isolated from the subject, and using a nanopore detection method to determine whether there is an excess of probe in the incubated sample (detection) or an excess of miRNA biomarker in the incubated sample (silencing), wherein detection is indicative of a reduced amount of the miRNA biomarker and that the subject has the disease.

### BRIEF DESCRIPTION OF THE DRAWINGS/FIGURES

FIG. 1A-D. (FIG. 1A) Schematic representation of a nanopore within a planar bilayer lipid membrane that separates two electrolyte filled compartments. Applying a constant voltage to the flow cell guides the passage of ions through the nanopore creating a measurable ionic current. (FIG. 1B) The i-t trace obtained from a voltage-driven ion-channel experiment where the constant flow of electrolyte ions (Io) via the pore is interrupted by the passage of molecules. These molecules appear as “events” with residual ion current Ir and residence time t. (FIG. 1C) OsBp labeling reaction: OsO4 and 2,2-bipyridine (bipy) have a low association constant, but their mixture adds to the C5-C6 double bond of pyrimidines and forms a stable conjugate. The addition of OsBp creates a chromophore that absorbs in the range of 312 nm where native nucleic acids do not absorb (see Examples). (FIG. 1D) Illustration of the concept behind the proposed diagnostic test. ssDNA and ssRNA traverse the nanopore and exhibit few counts because they traverse faster, compared to the device's relatively slow acquisition rate; ds nucleic acids are too big and do not traverse this nanopore. Despite being bulkier than ss native nucleic acids, osmylated ss nucleic acids traverse the pore, but more slowly compared to the device's acquisition rate and consequently produce numerous events. When an osmylated nucleic acid (probe) is added to a sample that contains its complementary nucleic acid (target), the probe and the target form a hybrid. When the target's concentration is equal or higher than the probe's concentration, the probe is hybridized. The probe is then prevented from traversing the pore, and few or no events are observed. The absence of target in the sample is evidenced by the numerous events produced by the probe while it freely traverses the pore.

FIG. 2A-B. Samples of tsv files, obtained by analyzing the fast-5 files using the OsBp_detect software. FIG. 2A, sample probe T8(9 OsBp), many events reported. FIG. 2B, sample is a mixture of d(CT)10:T8(9 OsBp)=1:1 hybrid, few events reported. Both samples in about 90% ONT buffer, nanopore experiments run for 1 h at −200 mV. Many more events are reported for the probe T8(9 OsBp) in the absence of its complement d (CT) 10 (sequenced listed in Table 6).

FIG. 3A-D. i-t recordings from two nanopore experiments ranging from 15 to 60 s. FIGS. 3A and 3B, Probe T8(9 OsBp), i-t recordings from two different channels. FIGS. 3C and 3D, mixture of d(CT)10:T8(9 OsBp)=1:1, i-t recordings from the same two channels as in FIGS. 3A and 3B. Vertical lines that cross the x-axis (0 pA) are instrument generated lines by voltage reversal, and not events. Recordings of the probe sample show multiple and deep events, while recordings from the hybrid sample show few and shallow events. Shallow events are attributed to molecules bumping at the pore aperture, without traversing the pore and they are not counted when selecting OsBp_detect parameter “All Ir/Io<0.6” (see FIG. 2).

FIG. 4. Graphical representation of all the processes involved in the miRNA measurement using the MinION platform. From left to right: (i) collection of the biospecimen, e.g., blood or urine; (ii) isolation of total RNA using a commercial kit; (iii) measurement of total RNA in the isolate using a NanoDrop spectrophotometer; and (iv) mixing of an aliquot from the RNA isolate with an aliquot of the probe complementary to the target miRNA, adding ONT buffer and conducting a MinION ion-conductance experiment (two experiments running simultaneously, shown here). The experiment measures the ion current (I) in picoAmperes (pA) as a function of time (t) in millisecond (ms). In practice, I is constant at Iₒ, which is the open nanopore ion current (Iₒ). When a single molecule traverses the nanopore, Iₒ is reduced to a new value, Iᵣ, because the molecule occupies the space that would have been occupied by the electrolyte that produces Iₒ. Ion current reduction (dip in this platform) lasts for a time, τ; both Iᵣ and τ depend on the molecular characteristics. The data were stored automatically as a fast5 file, which was subsequently analyzed by OsBp_detect (our software, see below). The analysis determines whether the free probe is in excess and detected (left on the scheme above) or if the probe is not detected because it is hybridized with the target (right on the scheme above). Notably, RNAs, including the target miRNA, traverse much faster than the probes, and they are not detected (bottom on the scheme above) due to the relatively slow acquisition rate of this platform.

FIG. 5. Data from Table 4, miRNAs per individual. miRNA copies were normalized to 16 ng/μL RNA content and then divided by the corresponding miRNA copy number in H6914 1^{st} lot. This double normalization yields Level 1.00 for all 4 miRNAs measured in H6914 1^{st} lot (not included in Table 4 or here). The rectangle across samples with y-axis ranging from 0.8 to 1.2 (average HL at 1.00 and RSD=0.2) includes 87% of the healthy data. The vertical dashed line separates cancer samples from healthy samples, whereas the horizontal dotted line at 1.5 HL is the target level which discriminates healthy samples from samples with elevated levels of miR-21, miR-375 and miR-141 biomarkers (see discussion). The data indicate that the test exhibits a 100% sensitivity and 100% specificity for each miRNA alone and for the miR-375 & miR-141 pair. Most importantly, this set of data validates each miRNA (miR-21, miR-375 and miR-141) as a cancer biomarker for breast, prostate, and pancreatic cancer indications.

FIG. 6: Examples of Yenos tests targeting let-7b taken from Table 3. Top figures illustrate detection experiments and bottom figures illustrate silencing experiments. Each figure contains a test, i.e., a set of 3 experiments with buffer (dotted line, open circles), followed by the 1st run of the sample which is the mixture of RNA with the probe (dashed line, solid triangles) and followed by a 2nd run of the same sample (solid line, solid circles). All 3 experiments conducted at −180 mV for 45 min. Analysis of the events by OsBp_detect typically yields two maxima, one early at Ir/Io=0.15 and a late at Ir/Io=0.3. As seen the buffer alone exhibits events at both maxima, but the Yenos probes traverse only at Ir/Io=0.15. Therefore, the presence of free probe is consistent with an increase at the early Ir/Io and/or a decrease at the late Ir/Io because there is a steady decrease of events due to the inactivation of the nanopores. Silencing experiments (bottom) often exhibit a markedly reduced number of events due to nanopore “shielding” as discussed in the text, while detection experiments (top) exhibit comparable counts but a reversed distribution with relatively more events at the early (Ir/Io)max=0.15 and fewer events at the late (Ir/Io)max=0.30. For a specific experiment with an x μL probe and y μL sample RNA, one calculates probe molecules P=x μL (probe concentration in fM)×600. If the experiment involved detection, then P>target miRNA molecules within the y μL aliquot. If the experiment involved silencing, then P<target miRNA molecules in y μL. It follows that the number of miRNA molecules per 1 μL of isolated RNA sample <or >P/y, depending on the experimental outcome.

FIG. 7: Examples of Yenos tests targeting miR-375 and miR-15b taken from Table 4. Top figures illustrate detection experiments and bottom figures illustrate silencing experiments. Each figure contains a test, i.e., a set of 3 experiments with buffer (dotted line, open circles), followed by the 1st run of the sample which is the mixture of RNA with the probe (dashed line, solid triangles) and followed by a 2nd run of the same sample (solid line, solid circles). All 3 experiments conducted at −180 mV for 45 min. Analysis of the events by OsBp_detect typically yields two maxima, one early at Ir/Io=0.15 and a late at Ir/Io=0.3. As seen the buffer alone exhibits events at both maxima, but the Yenos probes traverse only at Ir/Io=0.15. Therefore, the presence of free probe is consistent with an increase at the early Ir/Io and/or a decrease at the late Ir/Io because there is a steady decrease of events due to the inactivation of the nanopores. Silencing experiments (bottom) often exhibit a markedly reduced number of events due to nanopore “shielding” as discussed in the text, while detection experiments (top) exhibit comparable counts but a reversed distribution with relatively more events at the early (Ir/Io)max=0.15 and fewer events at the late (Ir/Io)max=0.30. For a specific experiment with an x μL probe and y μL sample RNA, one calculates probe molecules P=x μL (probe concentration in fM)×600. If the experiment involved detection, then P>target miRNA molecules within the y μL aliquot. If the experiment involved silencing, then P<target miRNA molecules in y μL. It follows that the number of miRNA molecules per 1 μL of isolated RNA sample < or >P/y, depending on the experimental outcome of detection or silencing.

### DETAILED DESCRIPTION

The terms defined immediately below are more fully defined by reference to the specification in its entirety. To the extent necessary to provide descriptive support, the subject matter and/or text of the appended claims is incorporated herein by reference in their entirety.

### Definitions

It will be understood by all readers of this written description that the exemplary aspects and embodiments described and claimed herein can be suitably practiced in the absence of any recited feature, element or step that is, or is not, specifically disclosed herein.

The term “a” or “an” entity refers to one or more of that entity; for example, “a probe,” is understood to represent one or more “probes.” As such, the terms “a” (or “an”), “one or more,” and “at least one” can be used interchangeably herein.

The term “and/or” where used herein is to be taken as specific disclosure of each of the specified features or components with or without the other. Thus, “and/or” as used in a phrase such as “A and/or B” herein is intended to include “A and B,” “A or B,” “A” (alone), and “B” (alone). Likewise, “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, technical and scientific terms used herein have the same meaning as commonly understood by one of ordinary skill in the art to which this disclosure is related. For example, unless otherwise specified, “complementary” base pairs refer to A/T, A/U, and G/C base pairing.

Numeric ranges are inclusive of the numbers defining the range. Even when not explicitly identified by “and any range in between,” or the like, where a list of values is recited, e.g., 1, 2, 3, or 4, unless otherwise stated, the disclosure specifically includes any range in between the values, e.g., 1 to 3, 1 to 4, 2 to 4, etc.

The headings provided herein are solely for ease of reference and are not limitations of the various aspects or aspects of the disclosure, which can be had by reference to the specification as a whole.

Where the term “about” is used, e.g., about 20%, it is understood that the value recited is also included. Further, where the term “about” precedes multiple numbers, for efficiency, all the numbers are qualified by “about,” even if each number is not directly preceded by “about,” unless explicitly defined otherwise. For example, “about 15%, 20%, 30%, 50%, or 75%” should be construed as “about 15%, about 20%, about 30%, about 50%, or about 75%,” etc. For example, “between about a 15% and 50%” should be construed as “about 15% and about 50%,” etc.

As used herein, the term “complementary” when referring to nucleic acid molecules is given its standard definition for complementary Watson-Crick base pairing as understood in the art.

The term “nucleic acid” is a well-known term of art and is used herein to include DNA and RNA. Unless otherwise specified, a “nucleic acid” molecule and “polynucleotide” can be used interchangeably. A nucleic acid can comprise a conventional phosphodiester bond or a non-conventional bond (i.e., an amide bond, such as found in peptide nucleic acids (PNA)). By “isolated” nucleic acid it is intended a nucleic acid molecule that has been removed from its native environment, such as total RNA obtained from a biospecimen using a suitable commercial RNA purification kit. Isolated polynucleotides or nucleic acids further include such molecules produced synthetically.

As used herein, the terms “intact” or “native” when referring to an oligonucleotide means that the oligonucleotide is not osmylated.

As used herein, a “biological sample” is one derived from a subject such as a human, animal, plant, bacteria, virus, fungus, or other type of multi-cellular or single-cellular life form. In certain aspects, the biological sample can be obtained directly from the subject, such as by drawing blood, collecting a urine sample, or a tissue or liquid biopsy. In certain aspects, the biological sample can be obtained indirectly, such as from biological evidence collected at a crime scene. In certain aspects, the biological sample is a bodily fluid such as blood, plasma, lymph, saliva, urine, amniotic fluid, spinal fluid, etc. In certain aspects, a biological sample is a fluid sample with components derived from a tissue or cells suspended, dissolved, in solution, reconstituted in, or the like, in the fluid sample. A “complex mixture” means a sample that comprises various components such as nucleic acids, proteins, carbohydrates, etc. and/or varied nucleic acid molecules.

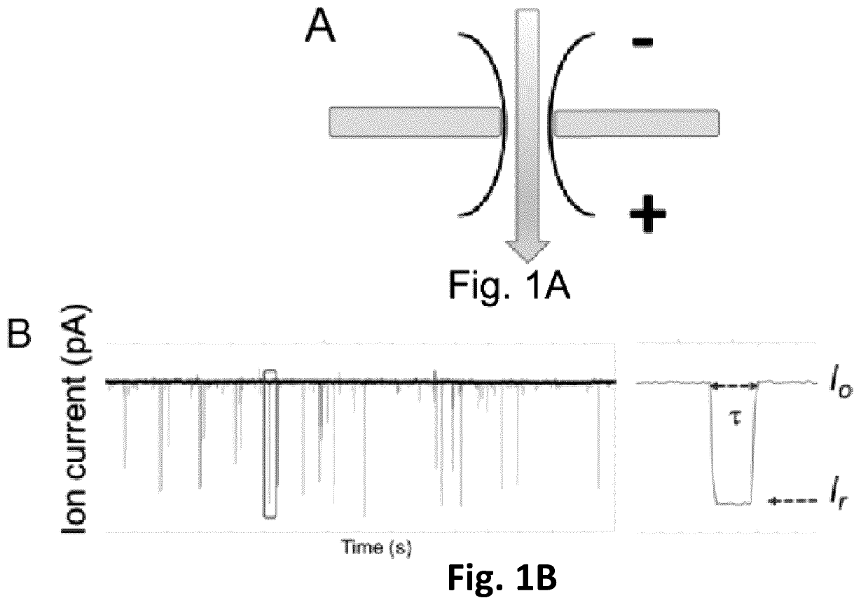

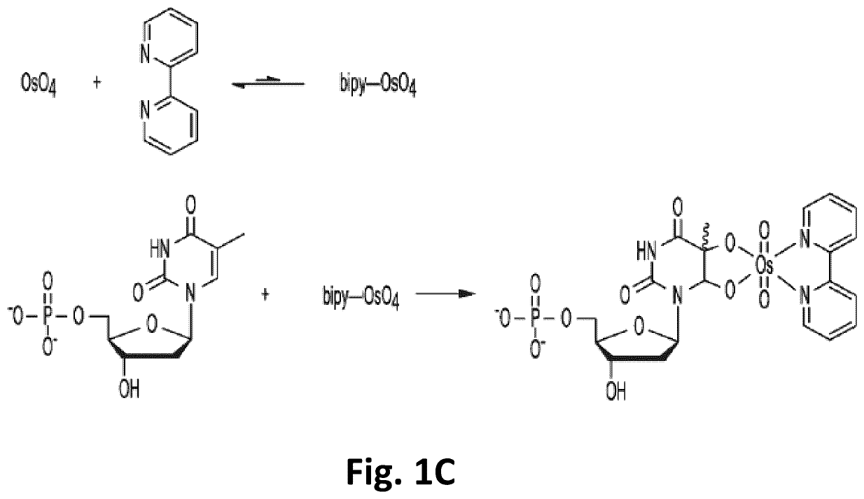

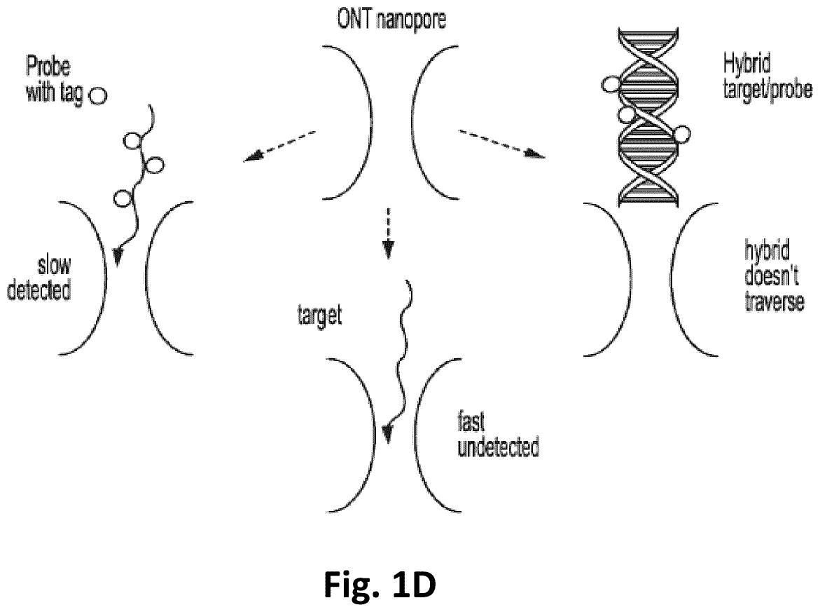

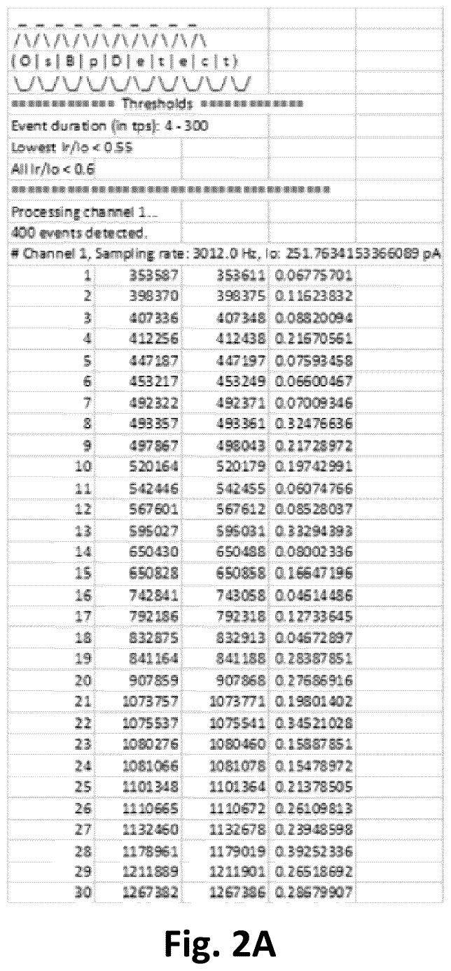

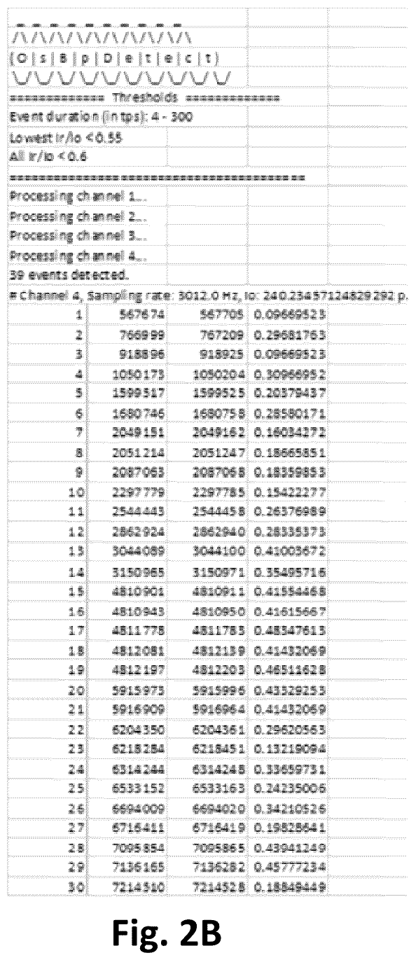

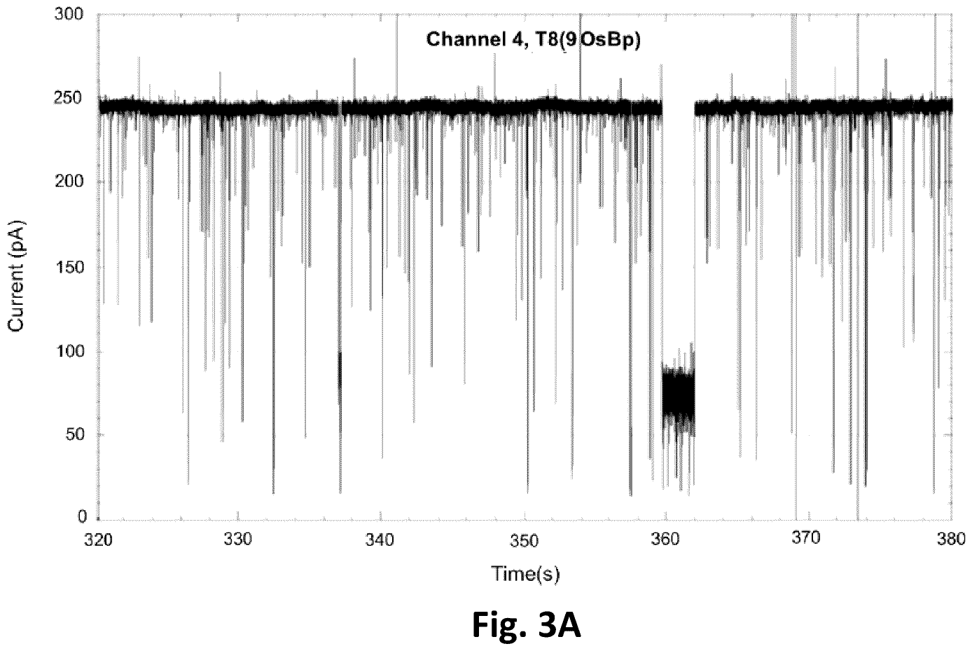

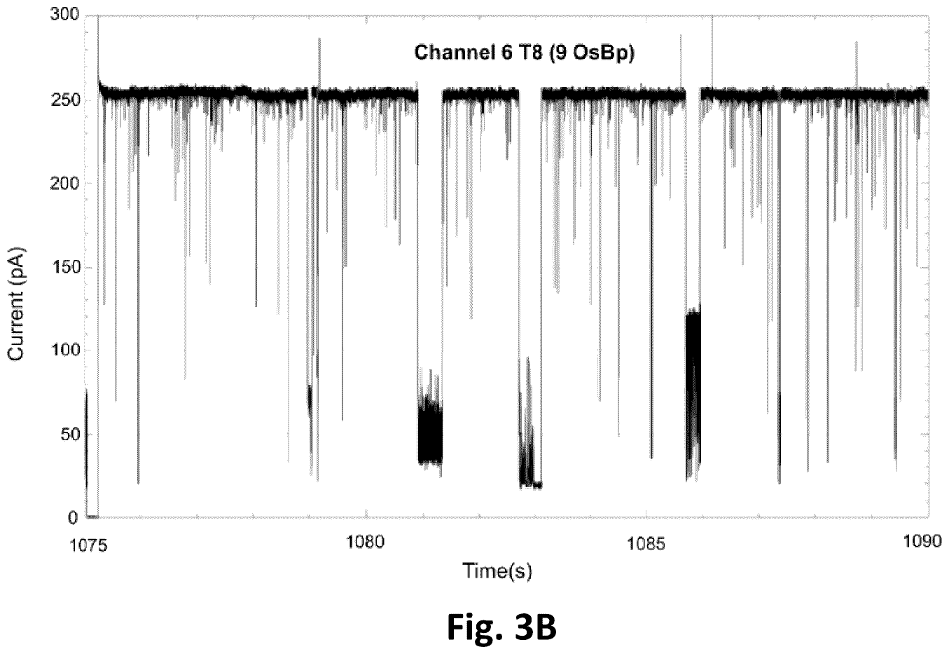

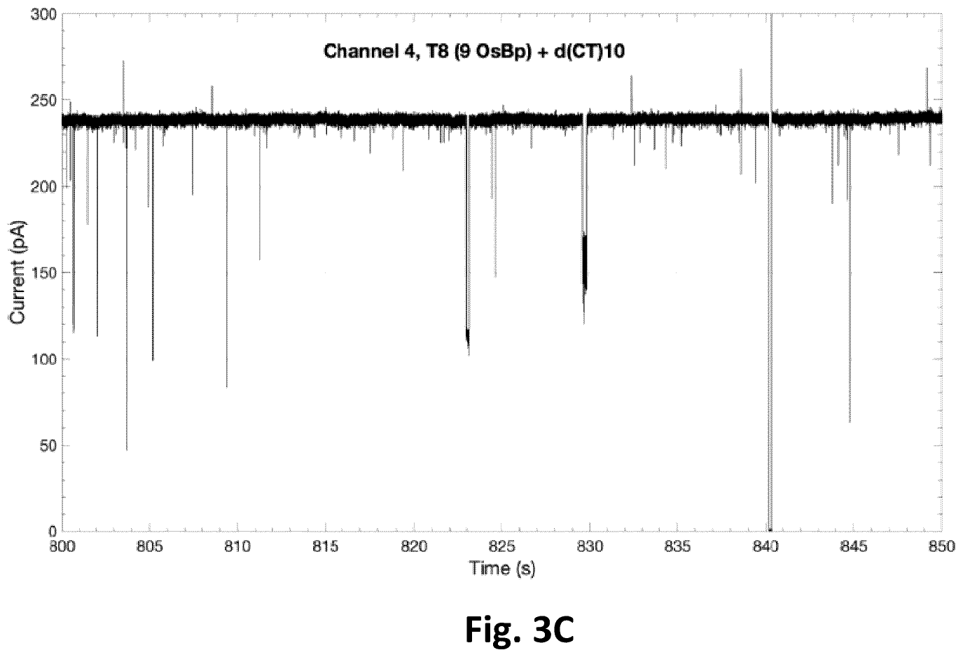

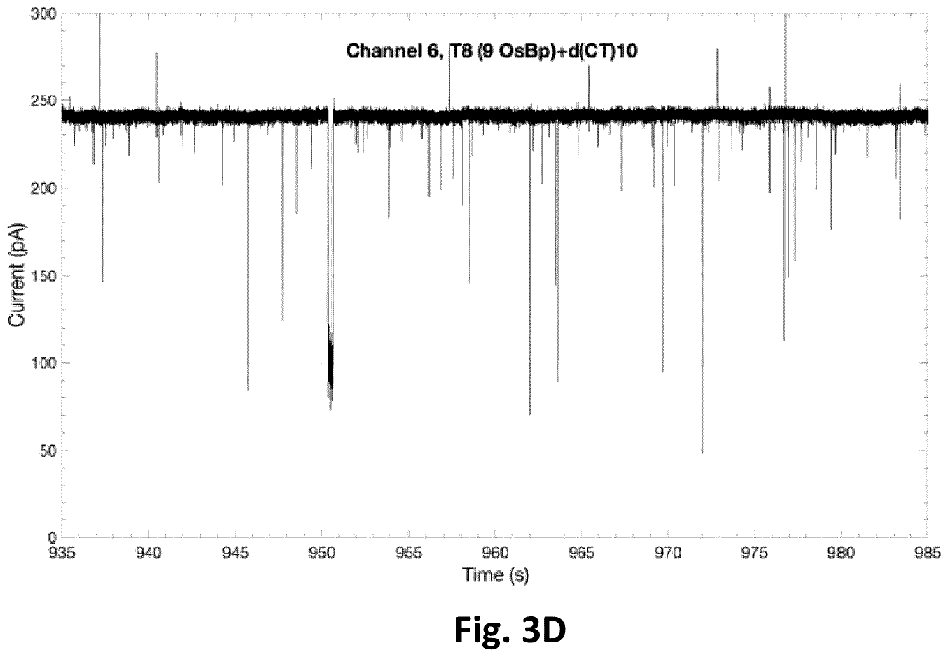

As used herein, an “event” or “count” is detected by applying a voltage across the two compartments of a nanopore device, leading to a constant flow of electrolyte ions (Io) via the pore, which is recorded as a function of time (i-t). The passage of a single molecule through the pore reduces Io to a lower level of residual ion current (Ir). This is recorded as an “event” with (Ir) and residence time (τ) (FIG. 1B).

As used herein, “accuracy” is the term used when there are only 2 numbers used to obtain an average. “Relative standard deviation” or “RSD” is used when there are 3 or more numbers to use to obtain an average (i.e., where n is equal to or more than 3).

As used herein, a “cancer indication” is a type of cancer such as, but not limited to, breast, pancreatic. or prostate cancer. If a biomarker is indicative of more than one cancer indication, then the biomarker may be considered a multi-cancer biomarker.

As used herein, “validating” a miRNA biomarker for a disease means that the normalized miRNA copy number obtained from the biospecimen of a subject can numerically classify this subject as having or not having the disease based on a collection of similarly normalized miRNA copy numbers obtained from control subjects and diseased subjects.

Validated microRNA Detection Assay

It is believed that the subject of this disclosure is unique in that the accuracy of the miRNA copy number measurement is “protocol-defined” by using two measurements to “bracket” the unknown miRNA copy number. Studies, including those disclosed herein, yielded a numerical value for the overexpression of miRNA cancer biomarkers compared to the healthy controls at about 2-fold. [Ban et al. (2023), Porzycki et al. (2018), Kanavarioti, 2022]. The approximate 2-fold overexpression is considered a small effect, difficult to assess with most amplification-based techniques. It may explain the observed disagreement among studies and the observation of overlap between cancer and healthy samples. To understand better the interplay between assay accuracy and miRNA over- or under-expression in disease, Table 1A and Table 1B below present the correlation between accuracy of the measurement and the corresponding x-fold overexpression of the biomarker (see Table 1A) or the corresponding x-fold underexpression of the biomarker (Table 1B). These correlations presume that the only significant parameter is disease or no disease. Other parameters, like age, gender, race, etc., if turn out to be significant, they will need to be addressed separately by limiting the tested population. For example, both tables illustrate that to achieve zero overlap between disease and healthy samples, the accuracy of the measurement for an overexpression of 1.85 must be better than 30%, i.e., numerically less than 30%. Similarly, an accuracy of 30% can only yield zero overlap if the overexpression is more than 1.85-fold. Notably, whether the specific miRNA biomarker is over- or under-expressed makes no difference to the accuracy of the measurement necessary to get reliable results (see Table 1B).

TABLE 1A
Correlation of measurement's accuracy and miRNA x-fold overexpression
to achieve zero overlap between healthy and diseased samples.

| | | | Normalized | | miRNA in |
|---|---|---|---|---|---|
| Accuracy of | Normalized | | disease | | disease/control, |
| measurement | control, | Average | overexpressed, | Average | Dn(norm)/Hc |
| (+/−) | range | control | range | disease | x-fold |
| 15% | 0.85 to 1.15 | 1.0 | 1.15 to 1.55 | 1.35 | >1.35 |
| 20% | 0.8 to 1.2 | 1.0 | 1.2 to 1.8 | 1.5 | >1.5 |
| 30% | 0.7 to 1.3 | 1.0 | 1.3 to 2.4 | 1.85 | >1.85 |
| 40% | 0.6 to 1.4 | 1.0 | 1.4 to 3.2 | 2.3 | >2.3 |
| 50% | 0.5 to 1.5 | 1.0 | 1.5 to 4.5 | 3.0 | >3.0 |
| 75% | 0.25 to 1.75 | 1.0 | 1.75 to 12.25 | 7.0 | >7.0 |

Footnote to Table 1A: Example of the calculations in the table are shown here for 20%. The average control=1.0. Range of normalized control for 20% accuracy defined as 1.0−0.2=0.8 (lower limit) and 1.0+0.2=1.2 (upper limit). Noted that Ratio of upper limit to lower limit equal to 1.2/0.8=1.5. The same Ratio of upper limit to lower limit applies to the range of normalized disease overexpressed, only that this lower limit (1.2) is equal to the upper limit of the Normalized control (1.2). Normalized disease overexpressed, upper limit=1.2×1.5=1.8, where 1.2 is the lower limit and 1.5 is the Ratio calculated above from the range for Normalized control. The other entries in this table are calculated in a similar manner.

TABLE 1B
Correlation of measurement's accuracy and miRNA x-fold under-expression
to achieve zero overlap between healthy and diseased samples.

| | | | Normalized | | miRNA in |
|---|---|---|---|---|---|
| Accuracy of | Normalized | | disease | | control/disease, |
| measurement | control, | Average | underexpressed, | Average | Hc/Dn(norm) |
| (+/−) | range | control | range | disease | x-fold |
| 15% | 0.85 to 1.15 | 1.0 | 0.63 to 0.85 | 0.74 | >1.35 |
| 20% | 0.8 to 1.2 | 1.0 | 0.54 to 0.8 | 0.67 | >1.5 |
| 30% | 0.7 to 1.3 | 1.0 | 0.4 to 0.7 | 0.55 | >1.8 |
| 40% | 0.6 to 1.4 | 1.0 | 0.3 to 0.6 | 0.45 | >2.2 |
| 50% | 0.5 to 1.5 | 1.0 | 0.18 to 0.5 | 0.34 | >2.9 |
| 75% | 0.25 to 1.75 | 1.0 | 0.036 to 0.25 | 0.14 | >7.1 |

Footnote to Table 1B: Small numerical differences between the two last columns in Table 1A and Table 1B are due to rounding up in calculations. For calculations see Note 1 under Table 1A, but here it is the lower limit of the Normalized control range that serves as the upper limit of the Normalized disease range, underexpressed.

Disclosed herein is an amplification-free assay, a detector suitable for nucleic acid trace measurements, and a protocol designed to deliver miRNA copies at an approximate +/−20% accuracy across all concentrations. Further disclosed are measurements using serum and urine samples, demonstrating the equivalency between serum and urine. The technology described here is well-positioned to overcome the inconsistencies in the miRNA field and revolutionize the current thinking in validating miRNA biomarkers.

Also disclosed herein is the fact that miRNA copy number is proportional to the RNA content of the sample. This fact is not consistent with prior studies of miRNA. For example: A sample of a healthy individual with isolated RNA content of 15 ng/μL was found to include 4,000 copies of miR-21. A sample of another healthy individual with RNA content at 30 ng/μL was found to include 8,000 copies of miR-21. These two results are perfectly comparable. Additional measurement of miR-21 in healthy individuals gave comparable results, demonstrating that miR-21 copy number is proportional to total RNA, and the correlation goes via 0.0. This means that when total RNA is zero, the miR-21 is also zero.

Aspects of this disclosure are drawn to methods of validating (as defined herein) a miRNA as a biomarker indicative of a disease, including cancer indications. In certain embodiments:

(a) The method comprises measuring a control miRNA copy number (e.g., H1) in a sample of isolated total RNA from a biospecimen of a control subject without the disease (isolated total RNA control sample). In certain embodiments, total RNA can be isolated from a biospecimen using a commercial RNA isolation kit or by long-established standard laboratory techniques. The total RNA content (e.g., in ng/μL) of a biospecimen is measured (e.g., using a NanoDrop UV-vis spectrophotometer). In certain embodiments, the RNA content is preferably more than or more than about 7 ng/μL. In certain embodiments, the RNA content is preferably less than or less than about 35 ng/μL. If the total RNA content measures more than about 35 ng/μL, it is preferably diluted with RNase free water to a concentration of at least about 7 ng/μL to not more than about 35 ng/μL. One of ordinary skill in the art will recognize that in certain embodiments, a biospecimen of a control subject can comprise the combined biospecimens from a plurality of control subjects. Thus, in certain embodiments, H1 is measured from a combined biospecimen of control subjects. The measurement of the miRNA copy number (in this or any miRNA copy number step of this disclosure) can be done using an osmylated probe that targets the miRNA and a nanopore detection method, as described in detail elsewhere herein (e.g., the Yenos test). The accuracy of the control miRNA copy number measurement is protocol-defined. “Protocol-defined” accuracy means that the measurement is the result of two experiments wherein one experiment shows that the probe is more than the target and the other experiment shows that the probe is less than the target. The average of these two experiments is then designated as the miRNA copy number. Notably if the two measurements are far apart, then the accuracy of the measurement is large. This is why the accuracy of the measurement is protocol-defined by designing the experiments so that they are different by a certain amount in one of the two components. In certain embodiments, the accuracy of the measurement is between +/−15% and +/−75%, between +/−15% and +/−50%, between +/−15% and +/−40%, between +/−15% and +/−35%, between +/−15% and +/−30%, between +/−15% and +/−25%, between +/−15% and +/−20%, between +/−16% and +/−24%, between +/−17% and +/−23%, between +/−18% and +/−22%, or between +/−19% and +/−20%. In certain embodiments, the accuracy of the measurement is +/−20% or about +/−20% (for example, to get accuracy=20%, one component needs to be at 67% in the second experiment).

(b) The method comprises repeating the measurement as above for one or more additional control samples (e.g., H2 to Hn). In certain embodiments, n (of Hn) is at least 3, 4, 5, 6, 7, 8, 9, or 10.

(c) The method comprises normalizing the miRNA copy numbers of the additional control samples H2 to Hn to the RNA content of the isolated total RNA control sample (from H1) and selecting the average miRNA copy number from H1 to Hn as a control miRNA copy number (Hc). Or, in certain embodiments whether explicitly stated or not, the control miRNA copy number Hc is the average miRNA copy number from H2 to Hn, for example if H1 is measured from a combined biospecimen of control subjects or otherwise not selected. Normalizing a miRNA copy number comprises (i) isolating the total RNA from a biospecimen (RNA isolate) and measuring the RNA content (X) of the RNA isolate (for example, in ng/μL), (ii) measuring the miRNA copies (Y) per selected unit (e.g., 1 μL) of the RNA isolate, to obtain Y miRNA copies per selected unit at an X RNA content, and (iii) mathematically adjusting Y to a Z RNA content, whereas Z is an arbitrarily selected control, to provide a normalized value (Ynorm), whereas Ynorm=Y*Z/X (where * is the multiplication sign and/is the division sign). In this description, Z is the isolated total RNA from control sample H1. The measured copy numbers H1 to Hn are normalized to a certain RNA content selected arbitrarily by the user, e.g., the RNA content of H1 (in which case, H1 is normalized against H1). The normalized H1 to Hn (or H2 to Hn) values can be used to select an average miRNA copy number as the control Hc. Certain embodiments comprise confirming that miRNA copy number from the combined biospecimen of healthy subjects fits within the correlation. If measured miRNA copies do not correlate with RNA content, then it would not be useful to try to make any conclusions from them. Similarly, the combined biospecimen of healthy subjects must fit in the correlation of the healthy subjects to enable miRNA validation. Hc can also be calculated, for example, by graphing the miRNA copy numbers H1 to Hn (or H2 to Hn) as a function of the corresponding isolated RNA in ng/μL, and assuming a statistically good correlation (e.g., 0.2<RSD<0.5), selecting an average miRNA copy number as the control Hc. Practically speaking, if the correlation is acceptable, any point within the range can be used as Hc. Hc is a copy number with a specific RNA content. For example, 3,540 copies miR-21 per 1 μL of isolated total RNA at 15.2 ng/μL. In certain embodiments, Hc is the average of the normalized H1 to Hn copy numbers with relative standard deviation (RSD) between 15% and 50%, between 15% and 45%, between 15% and 40%, between 15% and 35%, between 15% and 30%, between 20% and 30%, between 15% and 25%, between 15% and 20%, between 15% and 19%, between 15% and 18%, between 15% and 17%, between 16% and 20%, between 17% and 20%, between 18% and 20%, between 16% and 24%, between 17% and 23%, between 18% and 22%, and/or between 19% and 21%.

(d) The method also comprises measuring a disease miRNA copy number (D1), of the same miRNA measured from the control, in the isolated total RNA from a biospecimen of a disease subject confirmed to have the disease using the same osmylated probe used in to measure the control that targets the miRNA and the nanopore detection method. The accuracy of the disease miRNA copy number measurement is protocol-defined. In certain embodiments, the accuracy of the disease miRNA copy number measurement is protocol-defined to be comparable to and/or match that of the control sample measurement.

(e) The method comprises repeating the measurement as above for one or more additional disease samples (e.g., D2 to Dn). In certain embodiments, n (of Dn) is at least 3, 4, 5, 6, 7, 8, 9, or 10.

One of ordinary skill with understand that a biomarker could be overexpressed or underexpressed in comparison to healthy levels depending on the disease.

(f) For an overexpressed miRNA, the method comprises normalizing the miRNA copy numbers (e.g., D1 to Dn) of the disease samples to the RNA content of the isolated total RNA control sample (e.g., to calculate D1(norm) to Dn(norm)) and then dividing the disease sample normalized miRNA copy number values by the miRNA copy number of the control sample (Hc) to obtain a series of ratios of the normalized disease sample miRNA copy number to the control sample miRNA copy number, e.g., (D1(norm)/Hc to Dn(norm)/Hc).

(f) For an underexpressed miRNA, the method comprises normalizing the miRNA copy numbers D1 to Dn of the disease samples to the RNA content of the isolated total RNA control sample and then dividing the miRNA copy number of the control sample (Hc) by the disease sample normalized miRNA copy number values (D1(norm) to Dn(norm)) to obtain a series of ratios of the normalized disease sample miRNA copy number to the control sample miRNA copy number (Hc/D1(norm) to Hc/Dn(norm))

(g) The method concludes with making a determination whether the miRNA is a validated biomarker based on, or at least in part on, the ratios determined in the step above.

In certain embodiments, the disease is a cancer indication, and the miRNA is validated as a biomarker of this cancer indication. In certain embodiments, the miRNA is validated as a biomarker of multiple cancer indications, or the miRNA is validated as a biomarker of cancer.

One goal of developing validated assays indicative of a disease is early detection, while another is confirming a diagnosis. Thus, in certain embodiments, the biospecimen is collected from the disease subject at an early stage (I or II) of the disease. In certain embodiments, the biospecimen is collected ahead of treatment for the disease. It is noted that NIH/NCI has collected samples from “healthy” subjects once a year for several years and some of them later go on to develop a disease. Thus, it is contemplated that the method of the present disclosure can detect biomarkers of a developing disease and/or diagnose the disease years ahead of symptoms and disease development. Thus, in certain embodiments, a biospecimen is collected before a subject is diagnosed with a disease and the subject later develops and/or is diagnosed with the disease. While in certain other embodiments, the biospecimen is collected after treatment, for example, to monitor whether the treatment is effective.

In certain aspects, the validation of a biomarker is dependent on two parameters, one being the accuracy of the measurement of the biomarker and the other being the x-fold between control and disease for this biomarker. Practically speaking, the larger the x-fold, the lower the accuracy of the measurement can be and still allow validation. Optimal validation is defined when controls and diseased exhibit zero overlap. In certain embodiments, the validation of a biomarker using this assay (lower accuracy limit of +/−15%) will be technically possible if (D1(norm)/Hc to Dn(norm)/Hc)>1.35. For example, certain embodiments are drawn to a lower limit for RSD-0.15, which is based on the limitations of the techniques involved (e.g., pipetting and purity).

For example, if the RSD of the measurements H1 to Hn and D1 to Dn is less than or equal to 15% and the ratios in the series D1(norm)/Hc to Dn(norm)/Hc are more than 1.35, if the RSD of the measurements H1 to Hn and D1 to Dn is less than or equal to 20% and the ratios in the series D1(norm)/Hc to Dn(norm)/Hc are more than 1.5, if the RSD of the measurements H1 to Hn and D1 to Dn is less than or equal to 30% and the ratios in the series D1(norm)/Hc to Dn(norm)/Hc are more than 1.85, if the RSD of the measurements H1 to Hn and D1 to Dn is less than or equal to 40% and the ratios in the series D1(norm)/Hc to Dn(norm)/Hc are more than 2.3, or if the RSD of the measurements H1 to Hn and D1 to Dn is less than or equal to 50% and the ratios in the series D1(norm)/Hc to Dn(norm)/Hc are more than 3.0, then the miRNA is a validated overexpressed biomarker for the disease.

For example, if the RSD of the measurements H1 to Hn and D1 to Dn is less than or equal to 15% and the ratios in the series Hc/D1(norm) to Hc/Dn(norm) are more than 1.35, if the RSD of the measurements H1 to Hn and D1 to Dn is less than or equal to 20% and the ratios in the series Hc/D1(norm) to Hc/Dn(norm) are more than 1.5, if the RSD of the measurements H1 to Hn and D1 to Dn is less than or equal to 30% and the ratios in the series Hc/D1(norm) to Hc/Dn(norm) are more than 1.85, if the RSD of the measurements H1 to Hn and D1 to Dn is less than or equal to 40% and the ratios in the series Hc/D1(norm) to Hc/Dn(norm) are more than 2.3, or if the RSD of the measurements H1 to Hn and D1 to Dn is less than or equal to 50% and the ratios in the series Hc/D1(norm) to Hc/Dn(norm) are more than 3.0, the miRNA is a validated underexpressed biomarker for the disease.

Further, one of ordinary skill in the art will recognize that when multiple biological samples are involved, there can be variability and outliers in the data, and thus some amount of allowance for this can be permissible in validating a biomarker. For example, in any of the embodiments above, all of the ratios in the series D1(norm)/Hc to Dn(norm)/Hc (overexpression) or Hc/D1(norm) to Hc/Dn(norm) (underexpression) meet the specified criteria. But in other embodiments of any of the embodiments above, 50% or more, 55% or more, 60% or more, 65% or more, 70% or more, 75% or more, 80% or more, 85% or more, 90% or more, 95% or more, 96% or more, 97% or more, 98% or more, or 99% or more of the ratios in the series D1(norm)/Hc to Dn(norm)/Hc (overexpression) or Hc/D1(norm) to Hc/Dn(norm) (underexpression) meet the specified criteria.

One highly advantageous feature of the present disclosure is that in certain embodiments, the method does not require nucleic acid amplification as the detection method is sensitive enough to not require amplification. In certain embodiments, the method is sensitive enough to measure low abundance microRNAs contained in a 7 ng/μL total RNA isolate.

In certain embodiments, the subject (both disease and control) is an animal, such as a mammal, bird, reptile, amphibian, or fish. In certain embodiments, the subject is a mammal such as a cat, dog, horse, cow, pig, sheep, goat, llama, alpaca, rat, mouse, rabbit, guinea pig, etc. In certain embodiments, the subject is a human or a non-human primate. In certain embodiments, the biospecimen can be from a bodily fluid. In certain embodiments, the biospecimen is serum or urine. In certain embodiments, the biospecimen is derived from a tissue or organ.

In certain embodiments, the subject (both disease and control) is a plant. Thus, in certain embodiments, the biospecimen is collected from leaf tissue, callus, stem tissue, root tissue, flowers, pollen, oil, sap, and/or seed.

In certain embodiments, the miRNA is indicative of a disease that is a cancer indication or multiple cancer indications. In certain embodiments, the cancer indication is breast, prostate, or pancreatic cancer.

In certain embodiments, the validated miRNA biomarker can be used to detect a disease while the subject is asymptomatic, such as part of routine screening. In certain embodiments, the validated miRNA biomarker can be used to help confirm a diagnosis of a disease, such as at the initial onset of symptoms or other indications that the disease may be present. In certain embodiments, the validated miRNA biomarker can be used to monitor the effectiveness of a treatment and/or regression of the disease.

Another aspect of this disclosure is an elegant assay for determining whether the amount of a miRNA indicative of a disease is elevated or reduced based on a predetermined threshold amount. The assay can provide a simple readout and in certain embodiments can be used to detect a disease in a subject. In certain embodiments, the method comprises incubating a predetermined threshold amount of an osmylated-probe that binds to a miRNA biomarker of the disease with a sample of a specified amount of RNA isolated from the subject, and using a nanopore detection method to determine whether there is an excess of probe in the incubated sample (detection) or an excess of miRNA biomarker in the incubated sample (silencing), wherein silencing is indicative of an elevated amount of the miRNA biomarker and that the subject has the disease. In certain embodiments, the method comprises incubating a predetermined threshold amount of an osmylated-probe that binds to a miRNA biomarker of the disease with a sample of a specified amount of RNA isolated from the subject, and using a nanopore detection method to determine whether there is an excess of probe in the incubated sample (detection) or an excess of miRNA biomarker in the incubated sample (silencing), wherein detection is indicative of a reduced amount of the miRNA biomarker and that the subject has the disease. The predetermined threshold amount can be determined by the validation methods of this disclosure. For example, in certain embodiments, the method comprises incubating a predetermined threshold amount of an osmylated-probe that binds the miRNA biomarker with a sample of a specified amount of RNA isolated from the subject, and using a nanopore detection method to determine whether there is an excess of probe in the incubated sample (detection) or an excess of miRNA biomarker in the incubated sample (silencing), wherein silencing is indicative of an elevated amount of the miRNA biomarker and that the subject has the disease. Conversely, in certain embodiments, the method comprises incubating a predetermined threshold amount of an osmylated-probe that binds the miRNA biomarker with a sample of a specified amount of RNA isolated from the subject, and using a nanopore detection method to determine whether there is an excess of probe in the incubated sample (detection) or an excess of miRNA biomarker in the incubated sample (silencing), wherein detection is indicative of a reduced amount of the miRNA biomarker and that the subject has the disease.

In certain of embodiments, two or more miRNA biomarkers validated as disclosed herein are used to determine whether a subject has a disease. For example, in certain embodiments, the two or more miRNA biomarkers can be chosen from miR-15b, miR-21, miR-375, miR-141, and let-7b. In certain embodiments, the use of multiple validated biomarkers can be used to determine that a subject has cancer. In certain embodiments, the use of multiple validated biomarkers can be used to determine that the subject has a specific type of cancer. Consistent with foregoing, certain aspects provide for a kit comprising a predetermined threshold amount of an osmylated probe that binds to a miRNA biomarker indicative of a disease. In certain embodiments, the predetermined threshold amount of the osmylated probe is determined by the validation method of this disclosure. Such a kit could be a test kit where a user adds a sample to the kit to test whether the amount of relevant miRNA in the sample is above or below the threshold and thus indicative or not of a disease.

### Cancer Indications

One aspect of the present disclosure is drawn to a technology for detecting and quantifying trace nucleic acids with a protocol designed to yield copy number at about +20% accuracy across all concentrations. In certain embodiments, the microRNAs (miRNAs) let-7b, miR-15b, miR-21, miR-375 and miR-141 (see Table 2, Table 3, and Table 4) were measured from serum and urine samples. Detection and quantification were enabled via osmium-tagged probes and a nanopore-array detection device (e.g., MinION). The samples included the combined serum of healthy men purchased from Sigma-Aldrich (Cat #H6914), serum and urine from cancer patients, and urine from healthy subjects (IRB Advarra reviewed and approved protocol Pro00074065). Total RNA was isolated from the biospecimen using commercial kits and used as the miRNA source to determine miRNA copies per 1 μL of isolated RNA. miRNA copies normalized to the same RNA content (H6914, used here) appeared independent of age, gender, ethnicity, as well as of biospecimen, consistent with the use of a urine sample to replace a blood draw.

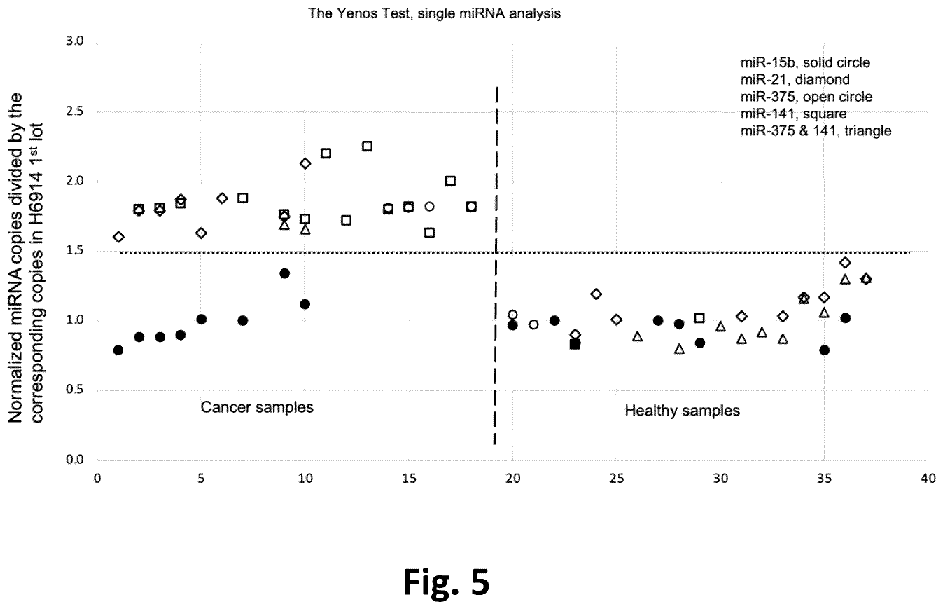

Data targeting let-7b is listed here in detail (see Table 3) to illustrate a strategy to validate any miRNA as a biomarker for a certain disease by comparing samples from subjects who were diagnosed with this condition with samples from subjects without the condition. In certain embodiments, measuring a miRNA copy number comprises at least two measurements: (i) one experiment where the probe copies exceed the target's copies yielding probe “detection” and (ii) another experiment where the probe copies are less than the target's copies and no probe is detected, referred to herein as “silencing.” Hybridization between target and probe is 1:1. The known concentration of the probe yields the number of probe copies included in each experiment. A few exploratory or confirmatory experiments are typically conducted. Among them two experiments that are “closest” to each other and where one is a detection and the other one is a silencing experiment, are selected. These two experiments provide an average probe count that “brackets” the miRNA copy number and is reported as such. It is up to the user to design the experiments so that the target copy number is obtained with high accuracy. Considering all the techniques involved here miRNA measurements with an approximate +/−20% accuracy or greater is possible. This accuracy may be achieved when one of the two components, RNA or probe, is 67% less in one of the two experiments, while the other component remains intact in both experiments (one experiment yielding detection and the other silencing). Table 3 shows experiments targeting let-7b and illustrates this unique experimental design and to show how it may be used to validate a miRNA target. Additionally, this approach demonstrated that miR-21, miR-375 and miR-141 are biomarkers for breast, prostate, and pancreatic cancers in contrast to miR-15b, which is not (see Table 4 and FIG. 5).

Due to this novel, protocol-defined, “bracketing” design of the experiments, a miRNA copy number is obtained with an accuracy as high as ±20%, which in turn yielded RSD ˜0.2 for a series of miRNA measurements from comparable samples and provided practically 100% sensitivity and 100% specificity in validating a miRNA as a biomarker. Specifically, let-7b copy numbers obtained from the combined serum H6914, the two healthy urine samples and the two pancreatic cancer serum samples are statistically indistinguishable (control group). These data (5 data points) yielded let-7b at 0.89 HL (RSD=0.21, HL stands for let-7b copies in the 1st lot of H6914 (Table 2)) and suggest that let-7b is not a biomarker for pancreatic cancer. On the contrary, the let-7b copy numbers from two prostate cancer urine samples and two breast cancer samples, one urine and one serum, measure 1.45 HL and 1.82 HL, and exhibit zero data overlap with the control group. Both these values are more than 1.5-fold higher compared to the let-7b control at 0.89 HL and suggests that let-7b is a biomarker for prostate and breast cancer (Table 3).

Additional experiments with healthy and cancer samples provided copy numbers for miR-15b, miR-21, miR-375 and miR-141 (Table 4). These copy numbers were first normalized to a control RNA content (16 ng/μl) and then to the corresponding miRNA from the 1st lot of H6914 (Table 2) to yield two groups with zero overlap, one averaging 1.01 HL with RSD=0.16 (40 counts: all miRNAs from healthy and miR-15b from cancer samples) and another averaging 1.83 HL with RSD=0.16 (28 counts: miR-21, miR-375, and miR-141 from cancer samples). These data illustrate that miR-15b is not a cancer biomarker in contrast to the observations with miR-21, miR-375 and miR-141. Additional samples are required to confirm these tentative validations. Notably, a p value of 1.6×10⁻²² was determined by Excel's t test for the combined 3 miRNA cancer biomarkers in the healthy vs. the cancer group (sample size 52). As a comparison, p values of approximately 0.001 were used for miR-141 measurements by droplet digital PCR (ddPCR) [Sequeira et al. (2023)]. which is currently considered the most accurate method. The remarkable discrimination disclosed herein with technology exhibiting zero overlap between the control and the cancer groups suggests the likelihood to validate miR-21, miR-735 and miR-141, each one separately, as biomarkers for breast, prostate, and pancreatic cancers, or as a multi-cancer biomarker with practically 100% sensitivity and 100% specificity.

These findings are groundbreaking and illustrate that miRNA copy numbers need to be normalized to the same RNA content, that serum and urine are interchangeable, and that miRNA biomarkers appear to be only 1.5 to 2-fold over-expressed in cancers. The latter observation necessitates miRNA validation using an analytical platform, like the one described herein, with a protocol-defined high accuracy for miRNA copy number determination close to +20% (see Table 1A and Table 1B).

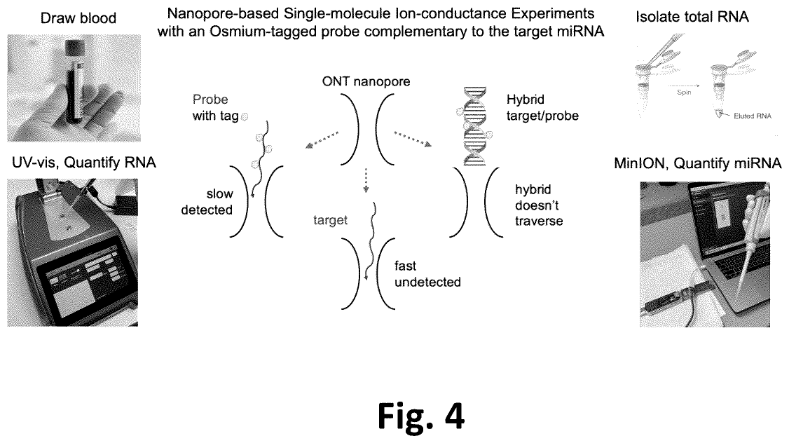

Nanopores have shown promise for trace measurements, and experimental platforms have been successfully used to quantify miRNAs [Kasianowicz et al. (1996); Butler et al. (2007); Maglia et al. (2010); Haque et al. (2013); Xi et al. (2016); Zahid et al. (2016); Henley et al. (2015); Ding & Kanavarioti (2016); Sultan & Kanavarioti (2019)]. For example, the MinION device from Oxford Nanopore Technologies (ONT) is used with a consumable flow cell which has 2048 nanopores embedded in it. This platform showcases 512 independent detection channels, 1 detection channel per 4 nanopores, and is promoted for sequencing long DNA/RNA. The ability of sequencing combined with DNA-barcoded probes has been exploited for the detection of biomarkers, including miRNAs [Cai et al. (2021); Koch et al. (2023)]. As an alternative, rolling circle reverse transcription was used to sequence miRNAs via the MinION [Maguire & Guan (2022)]. However, the lowest detection level of these techniques is in the picomolar range, which is 100 to 1000-fold higher compared to the miRNA levels in biological fluids, rendering these techniques unsuitable for miRNA testing. The MinION software, MINKNOW, reports the raw data, i.e., the ion current (i), as a function of time (t) and can be exploited for ion conductance (sensing) experiments, as described by us [Kang et al. (2020); Kanavarioti (2022); Sultan & Kanavarioti (2019)] and others [Quint et al. (2024)]. The inventor's earlier work illustrated that selective osmium tagging of an oligo yields a chemically stable probe that efficiently hybridizes with the complementary DNA, RNA, or miRNA target. These probes were shown to traverse size-appropriate proteins [Ding & Kanavarioti (2016)] and solid-state nanopores [Henley et al. (2015)], including the nanopores of the MinION platform [Kang et al. (2020); Kanavarioti (2022); Sultan & Kanavarioti (2019)] and see U.S. Pat. Nos. 11,111,527, 11,427,859, and 11,884,968, all of which are incorporated by reference herein in the entireties. Due to the bulkiness of the osmium tag, the translocation of the probe was markedly slower than that of intact RNA/DNA. While other nanopore platforms may detect and report all translocations, the MinION selectively detects probes over intact nucleic acids. This is the result of the relatively slow data acquisition rate of the MinION, at 3 data points/msec, which quantitatively detects optimized probes but misses most of the intact nucleic acids. In the absence of the target, the probe is free, traverses the nanopores and gets detected due to an increase in reported events over the background noise (see FIG. 1D and FIG. 4, scheme in the middle). In the presence of the target miRNA, the probe becomes hybridized, and the hybrid is too large to traverse the nanopore; the result is no probe detection or silencing. Target quantification is based on the 1:1 hybridization and the known probe concentration. The number of miRNA copies is determined from the average of two experiments, one that yielded probe detection and another that yielded no probe detection, or silencing [Kang et al. (2020); Kanavarioti (2022)]. To obtain the desired +/−20% accuracy, the two experiments must differ by approximately 67% in either probe or RNA. Typically, more than two experiments, are conducted to confirm a miRNA copy number determination.

ONT provides protocols for sequencing long RNAs/DNAs but not for ion conductance (sensing). The ONT provided software (MINKNOW) records the raw data from the ion conductance experiments, which are subsequently analyzed using a publicly available algorithm, OsBp-detect, developed by the inventor specifically for this application [Kanavarioti & Kang, (2020)]. While most studies report relative miRNA abundance, the technology of this disclosure measures actual miRNA copy numbers in the aliquot used for the nanopore experiment. Mitchell et al. (2008) reported miR-15b at ˜10,000 and miR-16 at ˜110,000 copies per 1 μL plasma from 3 individuals. The inventor recently reported miR-15b=8,855 and miR-16=105,125 per 1 μL H6914 serum [Kanavarioti (2022)]. This consensus represented the first demonstration of a MinION-based sensing assay. This agreement was partially fortuitous because the total RNA from the serum exhibited rather small sample-to-sample variation [Chomczynski et al. (2016)], and the RNA content from H6914 was comparable to the RNA content from the plasma of the individuals tested by Mitchell et al. (2008). 4 different lots of H6914 were obtained by purchase from Sigma-Aldrich and total RNA was isolated from each, which measured approximately 16 ng/μL. The copy numbers for 6 miRNAs were found to be reproducible (Table 2). This agreement is remarkable considering that (i) total RNA was isolated using different lots of the MONARCH RNA isolation kit from NEW ENGLAND BIOLABS, (ii) nanopore measurements were conducted by different analysts, (iii) chemically distinct probes were used, (iv) different experimental protocols were used, (v) different versions of MinION flow cells (R9 or R10) were used and (vi) different versions of the MINKNOW software were used.

For privacy reasons and to circumvent a blood draw, using urine as the miRNA source was explored, as miRNAs have been found to be relatively stable in urine [Mall et al. (2013)]. To assess directly whether miRNAs exhibit comparable copy numbers in serum or urine, the inventor initially purchased a set of 12 samples, matched serum and urine samples from the same donor, and 2 donors each for breast, prostate, and pancreatic cancer indications. Preliminary data supported the hypothesis that urine can replace a blood draw. However, the urine sample volume was only 1 mL, and 0.05 mL of total RNA was afforded at approximately 7 ng/μL concentration, which is at the lower limit of the technology of this disclosure. Additionally, the sample volume (0.05 mL) was not sufficient to run the number of experiments required to reach solid conclusions. The inventor did not pursue this type of matching serum and urine study further because of the striking equivalency between the miRNA copy numbers determined from H6914 serum and from the urine samples of healthy subjects (see Table 4). A recently developed slurry kit from NORGEN BIOTEK enables the isolation of total RNA from 5 to 10 mL of urine. Urine may contain up to 50-fold less RNA than serum. While 0.2 mL of serum provides 0.1 mL of isolated total RNA sufficient for multiple miRNA determinations, a much larger urine volume is necessary. miR-16 was found to be 12-fold more abundant in serum than was miR-15b [Mitchell et al. (2008); Kanavarioti (2022)]. Attempts to measure miR-16 in urine failed, suggesting that miR-16 may be under-expressed in urine compared to serum. The other five miRNAs were measured in both serum and urine samples (see Table 3 and Table 4).

A groundbreaking discovery was made when miRNA copies from the serum and from the urine (Urine1) of a healthy woman (A2) were found to be comparable to the corresponding H6914 miRNA copies (combined serum of healthy men, Table 2). A second urine sample from A2 (Urine2) confirmed these findings after normalization to the H6914 RNA content (footnote in Table 2). Notably, the three samples from A2 were collected many months apart and were of distinct RNA content. To the best of the inventor's knowledge this is the first demonstration of miRNA copy number equivalency between serum and urine.

TABLE 2
The number of miRNA copies measured per 1 μL of total RNA isolated from a biospecimen,
serum or urine (see footnotes for normalizing to the same RNA content).

| Bio- | H6914¹ | H6914¹ | H6914¹ | H6914¹ | | | |
|---|---|---|---|---|---|---|---|
| specimen | 1^{st} lot (HL) ² | 2^{nd} lot | 3^{rd} lot | 4^{th} lot | Serum A2³ | Urine1 A2³ | Urine2 A2^{3, 4} |
| Total RNA, | 16.0 | 16.5 | 15.9 | 14.3 | 20.7 | 16.8 | 27.4⁴ |
| ng/mL | | | | | | | |
| miRNA | | | | | | Copies | Copies |
| | | | | | | (+/− %) | (+/− %) |
| miR-16 | 210,250 | | | | | | |
| miR-15b | 17,710 | 16,716 | 17,687 | | 21,852 | 15,517 (7) | |
| let-7b | 12,150 | | | 8,668⁵ | | | 19,853 (37) |
| miR-21-5p | 10,494 | | 10,514 | | >2.0 × HL | 9,855 (14) | 21,352 (22) |
| miR-375- | 9,240 | 9,636 | 8,292 | | 1.5 to 2.0 × | | |
| 3p | | | | | HL | | |
| miR-141- | 6,096 | | 5,341 | 4,919⁵ | 1.5 to 2.0 × | 5,313 (12) | |
| 3p | | | | | HL | | |

¹H6914 is the combined serum of healthy men purchased from Sigma-Aldrich. miRNA copies measured from the first and second lots were reported earlier per 1 μL of serum [Kanavarioti, 2002] and are listed here per 1 μL of total RNA which equals to 2 μL serum. The accuracy values not listed here are all less than 0.2.
² HL stands for Healthy Level (H6914 1^{st} lot is the control/reference in this study).
³A2 is a healthy 72-years old woman tested multiple times over a period of 3 years (see Table 2 and Table 3); only the serum HW data were reported earlier [Kanavarioti, 2002]. A2 (woman) miRNA copies (6 measurements for 5 miRNAs) normalized to the RNA content of H6914 1^{st} lot are listed in Table 3 for both urine samples and illustrate the match with H6914 (men serum combined).
⁴The original Urine2 sample contained RNA at 82.3 ng/μL, which was diluted ⅓ with water before mixing with the probe. The number of miR-21 copies normalized to the RNA content in the H6914 1^{st} lot was 21,352 × 16/27.4 = 12,468. The number of copies of A2-related miR-21 normalized to the number of copies of miR-21 in the H6914 1^{st} lot was 12,468/10,494 = 1.19 (Table 4, 5^{th} row, in the Healthy Urinary section).
⁵Normalized to the 1^{st} lot of H6914 yields let-7b = 9,698 from 8668 × 16/14.3 and mir-141 = 5,503 from 4919 × 16.0/14.3, both within experimental error comparable to the other lots.

The samples tested in this study included H6914 (3^{rd} and 4^{th} lots), serum and urine from cancer patients, and urine from healthy subjects. The study was approved by Advarra IRB (see Methods). No formal follow-up was planned for the healthy group at this time. The cancer samples (breast, prostate and pancreatic) were purchased from two blood banks, Discovery Life Sciences and Tissue for Research, with subject requirements for early-stage diagnosis (I or II) and before treatment since miRNA levels may be influenced by disease stage and therapy. Instructions for urine collection were identical for women or men, healthy or diseased. The selection of an early disease stage ahead of treatment is consistent with our objectives to provide a validation strategy for miRNA biomarkers and develop a cancer screening test for asymptomatic individuals. The cancer samples were further selected to be as inclusive as possible regarding age, gender, and ethnicity (Table 5, Methods). The healthy urine samples were obtained from subjects who varied in gender and ethnicity and ranged from 22 to 72 years old.

TABLE 3
MinION experiments conducted targeting let-7b in healthy and cancer samples to demonstrate let-7b
copy number determination and assessing tentative validation of let-7b as cancer biomarker.

| | | | | Minion ion- | | | H6914 1st |
|---|---|---|---|---|---|---|---|
| | | | | conductance | | | lot 16.0 |
| | | | | experiment | | | ng/∞L |
| | | | isolated | | probe let-7b | probe | Normalized |
| | bio- | | total RNA | | 30 fM, 5.5 | copies per | probe |
| subject | specimen | condition | (ng/mL) | RNA (∞L) | Os (∞L) | 1 ∞L of RNA | copies |
| H1 | urine | healthy | 18.8 | 9.5 | 6.0 | 11,368 | 9,675 |
| ″ | ″ | ″ | ″ | 6.3 | 6.0 | 17,143 | 14,590 |
| H2 ⅓ | ″ | ″ | 27.4 | 6.0 | 6.0 | 18,000 | 10,499 |
| dilution | | | | | | | |
| ″ | ″ | ″ | ″ | 4.0 | 6.0 | 27,000 | 15,749 |
| ″ | ″ | ″ | ″ | 4.0 | 6.0 | 27,000 | 15,749 |
| ″ | ″ | ″ | ″ | 8.5 | 6.0 | 12,706 | 7,411 |
| H6914 | serum | ″ | 14.3 | 10.5 | 5.0 | 8,571 | 9,590 |
| 4th lot | combined | | | | | | |
| | men | | | | | | |
| ″ | ″ | ″ | ″ | 6.8 | 5.0 | 13,235 | 14,809 |
| ″ | ″ | ″ | ″ | 13.0 | 5.0 | 6,923 | 7,746 |
| SR16-690 | serum | PAN | 16.4 | 9.0 | 5.0 | 10,000 | 9,756 |
| | | cancer | | | | | |
| ″ | ″ | ″ | ″ | 6.0 | 5.0 | 15,000 | 14,634 |
| ″ B | ″ | ″ | 14.3 | 6.5 | 5.0 | 13,846 | 15,492 |
| ″ B | ″ | ″ | ″ | 13.0 | 5.0 | 6,923 | 7,746 |
| SR17-248 B | ″ | ″ | 21.0 | 4.5 | 5.0 | 20,000 | 15,238 |
| ″ | ″ | ″ | ″ | 7.5 | 5.0 | 12,000 | 9,143 |
| ″ | ″ | ″ | ″ | 9.5 | 5.0 | 9,474 | 7,218 |
| SR23-6022 | urine | PRO | 14.5 | 4.0 | 5.0 | 22,500 | 24,828 |
| | | cancer | | | | | |
| ″ | ″ | ″ | ″ | 5.0 | 4.0 | 14,400 | 15,890 |
| ″ | ″ | ″ | ″ | 8.0 | 4.0 | 9,000 | 9,931 |
| ″ | ″ | ″ | ″ | 4.0 | 4.0 | 18,000 | 19,862 |
| ″ | ″ | ″ | ″ | 5.3 | 5.0 | 16,981 | 18,738 |
| SR23-6028 | ″ | ″ | 12.4 | 10.0 | 4.0 | 7,200 | 9,290 |
| ″ | ″ | ″ | ″ | 6.0 | 4.0 | 12,000 | 15,484 |
| ″ | ″ | ″ | ″ | 4.7 | 4.0 | 15,319 | 19,767 |
| SR23-6016 | ″ | BRE | 43.7 | 4.0 | 6.0 | 27,000 | 9,886 |
| ¼ dilution | | cancer | | | | | |
| ″ | ″ | ″ | ″ | 2.7 | 6.0 | 40,000 | 14,645 |
| ⅛ dilution | ″ | ″ | 21.8 | 4.0 | 7.5 | 33,750 | 24,771 |
| ″ | ″ | ″ | ″ | 4.0 | 6.0 | 27,000 | 19,817 |
| ″ | ″ | ″ | ″ | 5.5 | 7.5 | 24,545 | 18,015 |
| CAN2 | serum | ″ | 17.1 | 5.5 | 6.0 | 19,636 | 18,373 |
| | matched | | | | | | |
| ″ | ″ | ″ | ″ | 4.0 | 6.0 | 27,000 | 25,263 |

| | | | | HL from | |
|---|---|---|---|---|---|
| | | | | H6914 1st | |
| | | | | lot, at | |
| | Normalized | | | 12,510 | |
| | probe | | | copies | Determination of |
| | copies/ | Ratio of late over | experimental | let-7b/ | subject's let-7b/ |
| subject | 12,150 | early (Ir/Io) max | result | let-7b HL | let-7b HL (+/−) |
| H1 | 0.80 | R = 3.5, 4.8, 9.0 | silencing | >0.80 HL * | |
| | | (increase × 2) | | | |
| ″ | 1.20 | R = 2.4, 1.2, 1.2 | detection | <1.20 HL * | 1.00 HL (0.20) |
| | | (decrease × 2) | | | |
| H2 ⅓ | 0.86 | R = 2.1, 3.9, 1.1 | Note 1 | | |
| dilution | | (increase × 1, | | | |
| | | decrease × 1) | | | |
| ″ | 1.3 | R = 6.7, 4.3, 1.9 | detection | <1.30 HL * | |
| | | (decrease × 2) | | | |
| ″ | 1.30 | R = 1.6, 1.5, 1.1 | detection | <1.30 HL * | |
| | | (decrease × 1) | | | |
| ″ | 0.61 | R = 4.1, 3.7, 5.4 | silencing | >0.61 HL * | 1.07 HL (0.37) |
| | | (increase × 1) | | | |
| H6914 | 0.79 | R = 2.2, 1.5, 2.3 | detection | <0.79 HL* | |
| 4th lot | | (decrease × 1) | | | |
| ″ | 1.22 | R = 3.5, 3.8, 2.4 | detection | <1.22 HL | |
| | | (decrease × 1) | | | |
| ″ | 0.64 | R = 0.97, 1.51, 1.02 | silencing | >0.64 HL * | 0.72 HL (0.08) |
| | | (increase × 1) | | | |
| SR16-690 | 0.80 | R = 1.2, 1.7, 1.2 | silencing | >0.80 HL * | |
| | | (increase × 1) | | | |
| ″ | 1.20 | R = 5.3, 2.0, 1.9 | detection | <1.20 HL * | |
| | | (decrease × 2) | | | |
| ″ B | 1.28 | R = 3.6, 1.9, 1.6 | detection | <1.28 HL | |
| | | (decrease × 2) | | | |
| ″ B | 0.64 | R = 0.99, 1.55, 0.81 | silencing | >0.64 HL | 1.00 HL (0.20) |
| | | (increase × 1) | | | |
| SR17-248 B | 1.25 | R = 1.55, 0.92, 1.55 | detection | <1.25 HL | |
| | | (decrease × 1) | | | |
| ″ | 0.75 | R = 1.06, 0.61, 0.74 | detection | <0.75 HL * | |
| | | (decrease × 2) | | | |
| ″ | 0.59 | R = 1.2, 2.3, 2.2 | silencing | >0.59 HL * | 0.67 HL (0.08) |
| | | (increase × 2) | | | |
| SR23-6022 | 2.04 | R = 3.7, 3.0, 1.0 | detection | <2.04 HL * | |
| | | (decrease × 2) | | | |
| ″ | 1.31 | severly reduced | silencing | >1.31 HL | |
| | | events | | | |
| ″ | 0.82 | severly reduced | silencing | >0.82 HL | |
| | | events | | | |
| ″ | 1.63 | severly reduced | silencing | >1.63 HL * | 1.82 HL (0.20) |
| | | events | | | |
| ″ | 1.54 | R = 1.6, 1.8, 1.3 | silencing | Note 1 | |
| | | (comparable) | | | |
| SR23-6028 | 0.76 | R = 0.7, 0.9, 1.7 | silencing | >0.76 HL | |
| | | (increase × 1) | | | |
| ″ | 1.27 | R = 1.9, 1.5, 2.7 | silencing | >1.27 HL * | |
| | | (increase × 1) | | | |
| ″ | 1.63 | R = 2.7, 1.7, 2.0 | detection | <1.63 HL * | 1.45 HL (0.18) |
| | | (decrease × 2) | | | |
| SR23-6016 | 0.81 | R = 1.5, 2.9, 2.3 | silencing | >0.81 HL | |
| ¼ dilution | | (increase × 2) | | | |
| ″ | 1.21 | R = 2.0, 2.3, 2.4 | Note 1 | | |
| ⅛ dilution | 2.04 | R = 6.6, 2.2, 3.6 | detection | <2.04 HL * | |
| | | decrease × 2) | | | |
| ″ | 1.63 | R = 1.7, —, 3.2 | silencing | >1.63 HL * | 1.77 HL (0.28) |
| | | (increase × 1) | | | |
| ″ | 1.48 | R = 1.1, 1.9, 3.2 | silencing | >1.48 HL | |
| | | (increase × 2) | | | |
| CAN2 | 1.51 | R = 3.8, 3.9, 4.3 | silencing | >1.51 HL * | |
| | | (increase × 1) | | | |
| ″ | 2.08 | R = 1.52, 0.75, 091 | detection | <2.08 HL * | 1.80 HL (0.28) |
| | | (decrease × 2) | | | |

In Table 3 above: PAN, PRO, and BRE stand for pancreatic, prostate and breast cancer, respectively. B at sample's ID stands for 2^{nd} total RNA isolation. Experiments with an asterisk in the column before the last column are the ones used to determine let-7b copy number and the others are confirmatory. Samples with RNA concentration higher than 35 ng/μL were diluted with Ambion water as shown on the first column. Assignment of an experiment as detection or silencing is based on the R factor which is the ratio of the event counts of the late (Ir/Io)max to the early (Ir/Io)max (see figures and methods for discussion and earlier work). One experiment in this technology is comprised of three nanopore experiments (45 min each at −180 mV). The first experiment is the baseline or control experiment and typically is an experiment with a buffer only or not. Then the mixture sample (RNA with probe) is loaded on the flow cell and run twice on the same flow cell as the control. The results of these three experiments are compared and R for each is determined in the order control, first run, second run. The R values of the 1^{st} and 2^{nd} runs are compared to the control and if, at least, one of them is a decrease, then the experimental result is detection. If, at least, one of them is increase, then the experimental result is silencing. If one of the runs is and increase and the other a decrease, the experiment is considered currently inconclusive, even though preliminary evidence suggests that this experiment yields miRNA copy number directly.

### Validation Strategy Illustrated by Targeting Let-7b in Three Cancer Indications.

For simplicity, a small number of representative samples were tested. In certain embodiments, validation can include at least 10 samples each from healthy subjects and subjects diagnosed with a certain disease. In certain embodiments, the sample from subject diagnosed with a certain disease is collected ahead of treatment. For example, at least 10, 20, 25, 30, 50, 75, or 100 samples each from healthy subjects and subjects diagnosed with a certain disease.

The first three samples in Table 3 are from healthy subjects, while the 4^{th} and 5^{th} samples are serum samples from patients with pancreatic cancer. Within the accuracy of the technology these five samples provided let-7b copy numbers that are statistically indistinguishable. To simplify the comparison, copy numbers are normalized to the H6914 1^{st} lot RNA content of 16.0 ng/μL (reference), and then divided by the let-7b copy number (12, 150) from this reference sample to give the HL number posted in the last column of Table 2. These five numbers (1.00, 1.07, 0.72, 1.00 and 0.67) give an average 0.89 HL with RSD=0.21. The tentative conclusion is that let-7b is not a pancreatic cancer biomarker which should be confirmed using additional samples.

The 6^{th} and 7^{th} samples are urine samples from patients diagnosed with prostate cancer with let-7b copy number at 1.82 and 1.45 HL (HL from H6914 1^{st} lot). Compared to the control 0.89 HL, the 6^{th} sample is 2.0-fold, and the 7^{th} sample is 1.6-fold higher; both are over expressed by more than 1.5-fold.

The 8^{th} and 9^{th} samples are from breast cancer patients, one obtained from a urine and the other from a serum sample with let-7b copy numbers 1.77 HL and 1.80 HL. Both measurements are 2.0-fold over expressed compared to the control 0.89 HL. These data suggest that 1.5 may serve as a threshold, whereby a miRNA level above 1.5 HL suggests cancer detection and miRNA level below 1.5 HL indicates absence of cancer. Below are more experiments conducted with additional miRNAs, where the threshold of 1.5-fold is also applicable.

TABLE 4
Measured miRNA copies were normalized to 16 ng/mL (H6914 1^{st} lot) and then
divided by the copies of the corresponding miRNA from the 1^{st} lot of H6914
(see, Table 2). Values in columns 4 through 8 are in HL units.

| | | | miRNA targets | | | | |
|---|---|---|---|---|---|---|---|
| | | isolated | | | | | miR-375 + |
| ID | indication | RNA, ng/mL | miR-15b | miR-21 | miR-375 | miR-141 | miR-141 |
| Cancer serum | | | | | | | |
| CAN7 | breast | 6.9 | 0.79 | 1.60 | | | |
| CAN9 | ″ | 9.1 | 0.89 | 1.79 | | 1.80 | |
| CAN4 | prostate | 12.0 | 0.88 | 1.79 | | 1.81 | |
| CAN6 | ″ | 8.0 | 0.90 | 1.87 | | 1.84 | |
| SR16-690 | pancreatic | 16.4 | 1.01 | 1.63 | | | |
| | | | | 1.88 | | | |
| SR17-248 | pancreatic | 14.4 | 1.00 | | | 1.88 | |
| Cancer urine | | | | | | | |
| SR23 6016 | breast | 174.6 | 1.34 | 1.75 | | 1.76 | 1.69 |
| SR23 6017 | ″ | 88.8 | 1.12 | 2.13 | | 1.73 | 1.66 |
| | | | | | | 2.20 | |
| SR23 6018 | breast | 16.1 | | | | 1.72 | |
| | | | | | | 2.25 | |
| SR23 6022 | prostate | 15.3 | | | 1.81 | 1.80 | |
| SR23 6028 | ″ | 13.3 | | | 1.81 | 1.82 | |
| SR23 6023 | ″ | 18.5 | | | 1.82 | 1.63 | |
| | | | | | | 2.00 | |
| SR23 6033 | pancreatic | 13.4 | | | 1.82 | 1.82 | |

| Healthy urine | 7.5, 22.9 | 0.97 | | 1.04 | | |
|---|---|---|---|---|---|---|
| | 21.7 | | | 0.97 | | |
| | 9.5 | 1.00 | | | | |
| | 16.8, 82.3 | 0.84 | 0.90 | | 0.83 | 0.84 |
| | | | 1.19 | | | |
| | 8.7 | | 1.01 | | | |
| | 16.5 | | | | | 0.89 |
| | 12.2 | 1.00 | | | | |
| | 57.4 | 0.98 | | | | 0.80 |
| | 163.0 | 0.84 | | | 1.02 | |
| | 11.7 | | | | | 0.96 |
| | 25.6 | | 1.03 | | | 0.87 |
| | 14.5 | | | | | 0.92 |
| | 16.4 | | 1.17 | | | 1.16 |
| | 13.8 | 0.79 | 1.17 | | | 1.06 |
| | 12.4, 15.7 | 1.02 | 1.42 | | | 1.30 |
| | 14.7 | | 1.30 | | | 1.31 |

In Table 4 above, the cancer serum samples (second, third and fourth row entries) were also used in an earlier study [Kanavarioti, 2002], but only miR-15b was measured earlier. The same RNA isolate was used for the additional miRNAs measured here. Multiple entries of isolated RNA for healthy samples correspond to separate collections. When the amount of isolated RNA exceeded 35 ng/μL, the RNA was diluted with Ambion water ½ or ¼ before mixing with the probe. The last column reports the results of a single experiment in which two miRNAs were simultaneously targeted. Experiments that yielded silencing or detection but were not followed by the corresponding test to lead to a miRNA copy number determination were not included here but the results are consistent with the measured miRNA copy numbers.

The number of miR-15b copies in the serum of H6914 and in the sera of healthy individuals and cancer patients was found to be directly proportional to the RNA content in the range of 9.1 to 20.7 ng/μL RNA [Kanavarioti (2022)]. This observation was reported earlier (8 samples, 4 healthy and 4 diseased), confirmed here, and extended by including serum and urine data (13 new samples, 8 healthy and 5 diseased) in the range of 6.9 to 174.6 ng/μL RNA (Table 4). These data suggest that miR-15b is not a cancer biomarker, in agreement with the findings of Mitchell et al. (2008). The observed independence on age, sex, or ethnicity is in bold contrast to studies that report large data variation and attribute it to age, sex, ethnicity, and other parameters. The data of this disclosure irrevocably established that miRNA copies must be normalized to the same RNA content, which is currently not common practice. There is literature consensus that miR-21, miR-375 and miR-141 are cancer biomarkers and overexpressed in cancer samples compared to healthy samples. In agreement with selected studies [Porzycki et al. (2018)], the inventor's earlier report indicated an approximate 2-fold overexpression which was confirmed here (Table 4). The less than 2-fold overexpression appears too small of a difference to be quantified by other technologies, such as microarray-based or amplification-based assays.

Normalization to the same RNA content (16.0 ng/μL in H6914, 1^{st} lot) for all tested miRNAs yielded copy numbers independent of age, sex, ethnicity, and cancer indication (breast, prostate, or pancreas), as well as biospecimen, suggesting that a urine sample may replace a blood draw. In certain embodiments, normalization is done by dividing the miRNA copies of a sample by the corresponding miRNA copies from a reference (e.g., H6914 (1st lot)). Further normalization yielded two groups with zero overlap, one averaging 1.01 HL with RSD=0.16 (40 counts: all miRNAs from healthy samples+miR-15b from cancer samples) and another averaging 1.83 HL with RSD=0.16 (28 counts: miR-21, miR-375, miR-141 from cancer samples) (Table 4 and FIG. 5). On first inspection, the data yielded 100% sensitivity and 100% specificity, but the sample size was small, and whether the healthy subjects would remain free of cancer and for how long was not assessed. Notably, a p value of 1.6×10⁻²² was determined by Excel's t test for the combined 3 cancer biomarkers in the healthy vs. the cancer group (sample size 52). As a comparison, p values of approximately 0.001 were exploited for miR-141 measurements by ddPCR, which is currently considered the most accurate method [Sequeira et al. (2023); Hindson et al. (2013)]. The unprecedented discrimination observed between healthy samples and cancer samples from breast, prostate and pancreatic patients may validate each of these three miRNAs as multi-cancer biomarkers. Specifically, the overexpression of miR-21 [Jenike & Halushka (2021)] and miR-141 [Mitchell et al. (2008)] has been associated with numerous cancer indications in addition to breast, prostate and pancreatic. Further testing of this set of miRNAs with samples from additional cancer indications may illustrate their usefulness as a multi-cancer biomarker. As long as a ss nucleic acid biomarker, such as miRNA, is elevated by 80% or more between diseased and healthy samples, our protocol-defined +/−20% accuracy in miRNA copy number determination is compatible with a 1.5 HL threshold to assign an unknown sample as healthy or cancerous. The data in Table 4, in conjunction with the miRNA studies conducted worldwide during the last 25 years, suggest that elevated levels of miR-21, miR-375 and/or miR-141 in the serum or urine of an individual are concerning and warrant consultation with a doctor, just as high fever would.

The nanopore technology used in certain embodiments for trace nucleic acid detection and quantification is unique and has no similarities to currently used assays; it was developed and optimized earlier [Kang et al. (2020); Kanavarioti (2022); Sultan & Kanavarioti (2019)] and see U.S. Pat. Nos. 11,111,527, 11,427,859, and 11,884,968, all of which are incorporated herein in the entireties. To the best of the inventor's knowledge, no other analytical assay yields measurements with a protocol-defined accuracy; here, set up at +/−20%. Each process involved in this assay is described and outlined here. Total RNA (0.1 mL) was isolated from the serum (0.2 mL) using a MONARCH kit from NEB, and total RNA (0.05 mL) was isolated from the urine (5 mL) using a NORGEN slurry kit. Total RNA in ng/μL was measured using a DS-11 NANODROP spectrophotometer from DENOVIX, Inc. For accuracy reasons, in certain embodiments, isolated total RNA contains more than 7 ng/μL RNA and an absorbance ratio at 260 nm vs. 280 nm, A260/A280, better than 1.6. Capillary electrophoresis (CE) analysis was used, as described earlier [Kanavarioti (2022)], to confirm the dramatic variation observed in the total RNA isolated from individuals.

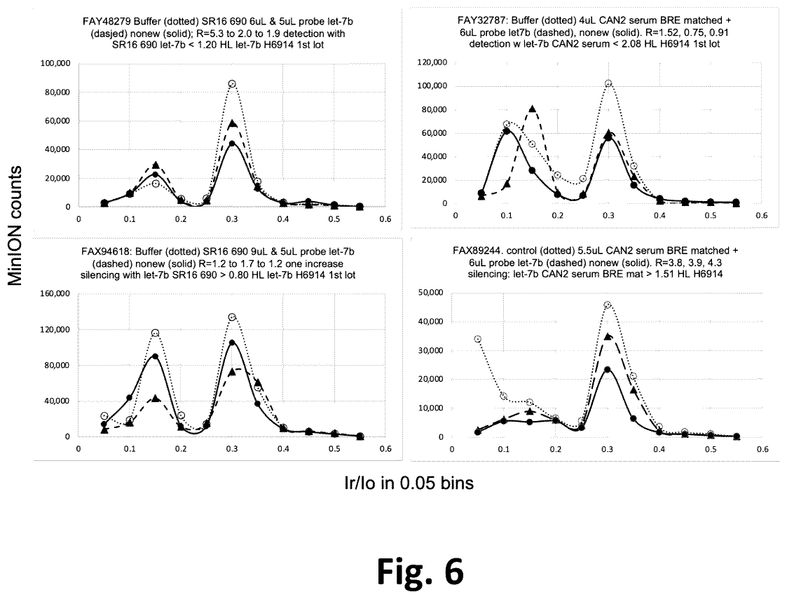

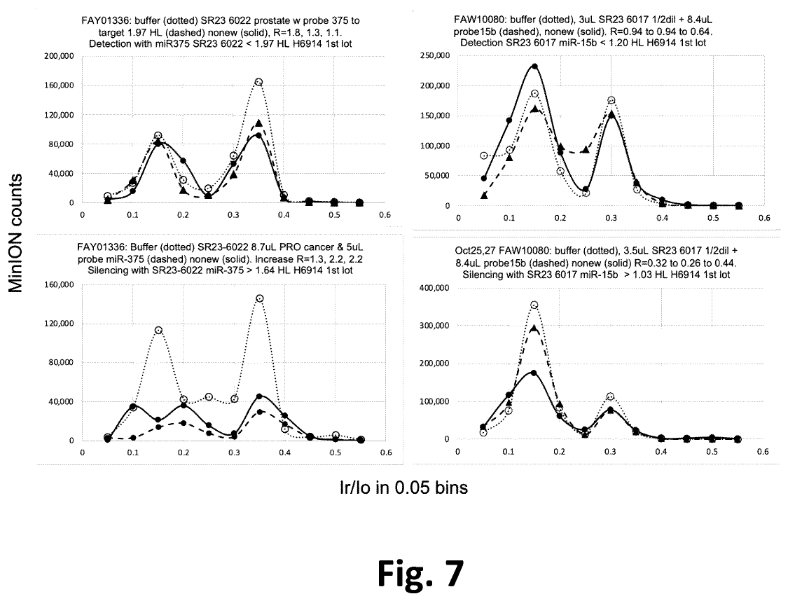

In certain embodiments, samples for the nanopore experiments were prepared by mixing a few μL of the isolated total RNA with a few μL of the probe complementary to the target miRNA. The mixture was stored at −20° C. overnight to ensure complete hybridization, and 75 μL of filtered ONT buffer was added to this mixture immediately before loading it onto the MinION flow cell. The nanopore experiment was conducted for 45 min at −180 mV. The flow cell was allowed to rest for 15 min, and a second run was conducted under the same conditions. The MINKNOW software runs the experiments and produces a fast5 file (the ion current (i) with time (t)), which is subsequently analyzed using OsBp-detect [Kanavarioti & Kang, github]. The latter yields a tsv file that can be opened in Excel. The data are grouped in the form of a histogram with 0.05 bins (FIG. 6 and FIG. 7). An experiment using buffer only, instead of a sample, primes the flow cell and serves as a control for the experiment with the RNA/probe sample. If the test with the mixture of RNA and probe is determined to be a silencing one, then the next test may be designed using the same aliquot of probe but only 67% of the RNA aliquot. If this second test was determined to be a detection experiment, then miRNA copies per μL of RNA sample is determined from the average of the probe copies per μL of RNA sample from the two test samples. Typically, more than two tests are necessary before finding the set, one detection and the other silencing, that fulfills the accuracy requirement. Due to the low throughput of the assay and the approximately 15-hour life span of the flow cells, not all four miRNAs were measured in every sample. This platform may be further optimized by developing a buffer optimal for ion conductance experiments to replace the currently used buffer. Buffer optimization may enable direct quantitation of miRNA targets from a single experiment by suppressing and/or controlling background noise.

It is contemplated that a solid-state nanopore array will be a more robust and cost-efficient alternative to proteinic MinION nanopores. The technology disclosed herein could be implemented in other nanopore arrays with no or minor optimization, as shown earlier using α-hemolysin [Ding & Kanavarioti (2016)] and silicon nitride nanopores [Henley et al. (2015)]. The only requirement for this assay to work is that the width of the nanopore permits the translocation of ss nucleic acids and prevents the translocation of ds nucleic acids. Interestingly, the “bulkier” probe traverses nanopores of the same width as unlabeled (intact) nucleic acids.

One of ordinary skill in the art will recognize that this disclosure is not limited to the embodiments of the five miRNAs measured in the above exemplary discussion. The probe is a DNA oligo complementary to the target sequence. Osmium tagging is straightforward, and the resulting probe is stable and characterizable. The design of the probe is general and has been optimized for efficient hybridization with a DNA or RNA target and for nanopore detection. Probes for a limited number of human miRNAs have been manufactured and tested; miRNAs from other species could also be targeted. The technology is not limited to miRNA quantification. Circulating tumor DNA (ctDNA), circular nucleic acids, and practically any ss nucleic acid of a known partial sequence of interest may be detected and quantified. Using liquid biopsies as samples is noninvasive, but this assay is not limited to liquid biopsies. Due to the availability of commercial kits for total RNA isolation (including miRNAs) from practically any tissue or organ, the latter can be used as a source for miRNA detection and quantification.

Implementation of a multicancer test will not require extensive testing, such as the ones described in Table 3 necessary for miRNA validation studies. Instead of determining miRNA copy number, the concept of threshold value may be implemented as follows: Considering a miRNA biomarker, like the ones studies here where the HL level is close to 1.0 and the cancer level is close to 1.8, both at ˜0.2 RSD, an experiment designed to target a miRNA level at ˜1.5 HL, called threshold, should yield detection with healthy samples and silencing with cancer samples or samples from asymptomatic individuals with elevated miRNA (Table 3, Table 4, and FIG. 5). It is proposed that elevated miRNA levels, in two out of three tested miRNA cancer biomarkers, indicate miRNA dysregulation which may be associated with the onset and/or presence of cancer and represent a warning sign for the tested individual. The number of false-positive and false-negative results from a tentative multi-cancer test targeting ˜1.5 HL should be practically nonexistent, as shown in Table 3, Table 4 and FIG. 5. This is because a single miRNA test (the Yenos test) includes a baseline experiment and two runs using the same sample. A second test for an additional miRNA will also include one baseline and a second sample run twice. Six separate experiments will be testing for two miRNA-related cancer biomarkers at ˜1.5 HL threshold, and the results should lead to one conclusion, namely, whether the biomarkers were detected or silenced. This approach was exploited by testing consented individuals and showed that it works (data not included). One consented individual was a breast cancer survivor and her miRNA levels, tested 4 years later, were comparable to the healthy control which suggests that miRNA levels recover. Another individual who tested positive for cancer in the Yenos urine test, took the GALLERI test which also exhibited cancer detection, confirming the Yenos test. Notably the GALLERI test evaluates DNA methylation, while the Yenos test evaluates miRNA overexpression, making these two tests completely independent. It turned out that all four miRNAs tested exhibited a comparable overexpression in the cancer samples (1.5 HL). Additional miRNAs may exhibit a comparable or different overexpression and if overexpression is at least 1.8-fold, our technology with the protocol-defined RSD ˜0.2 will discriminate healthy from diseased samples.

Table 4 lists the results of the experiments in which two miRNAs, i.e., miR-375 and miR-141, were simultaneously targeted using the two corresponding probes. Targeting two miRNAs in one experiment yielded one copy number for both miRNAs, which reduced the number of experiments twofold, and could still serve as a screening test using an appropriate threshold value, as outlined above. FIG. 5 illustrated the successful use of the combined miRNAs to discriminate cancerous from healthy samples. This approach can be used only when the two targeted miRNAs exhibit similar copy numbers, as is the case for miR-375 and miR-141. This limitation is due to the inability of nanopores to discriminate one probe from another with the current probe design (Table 5). Earlier work with a-hemolysin [Ding & Kanavarioti (2016)] and solid-state nanopores [Henley et al. (2015)] illustrated that more osmium tags yielded deeper translocations with earlier (Iᵣ/Iₒ)ₘₐₓ. Adding another 2 to 3 osmium tags to the current 5 may yield the desired discrimination and enable individual miRNA testing in a single test targeting two miRNAs.

Thus, this disclosure is drawn to the implementation of a novel analytical platform for the detection, quantification, and validation of miRNA cancer/disease biomarkers, such as from liquid biopsies, with an experimental design that yielded data with a protocol-defined ˜20% RSD. This technology combines single-molecule ion-conductance experiments using the MinION functionality with expertly optimized and validated probe design. A general validation strategy is outlined here applicable to any potential ss nucleic acid biomarker. The copy numbers of five miRNAs, let-7b, miR-15b, miR-21, miR-375 and miR-141, were measured from healthy and cancerous samples, and the results revealed equivalency between the serum and urine samples after normalization to the same RNA content. In contrast to miR-15b, which appears to be unrelated to breast, prostate and pancreatic cancers, and let-7b which is not over-expressed in prostate cancer, the other three miRNAs were elevated in cancer samples compared to healthy controls, in agreement with the findings of multiple studies conducted during the last 25 years. The 1.8-fold overexpression of these cancer biomarkers agrees well with the overexpression observed earlier in a prostate cancer study [Porzycki et al. (2018)]. Also, the 1.8-fold overexpression of miR-21 is in excellent agreement with the 1.7 overexpression observed in a lung cancer study [Ban et al. (2023)]. Surprisingly, the normalized copy number of each of the five miRNAs appears to be independent of age, gender, and ethnicity, in bold contrast to the variability seen by current miRNA quantification platforms. Comparison to the corresponding miRNA copy number, as determined in the combined serum of healthy men (H6914 from Sigma-Aldrich), grouped the data into healthy and cancer samples with no data overlap, i.e., 0 false negatives and 0 false positives, yielding sensitivity, specificity, positive predictive value (PPV) and negative predictive value (NPV), all equal to 1. This unprecedented discrimination validated each miRNA separately, miR-21, miR-375 and miR-141, as cancer biomarkers. The technology merits further testing in a larger sample size and for other indications in addition to the breast, prostate and pancreatic cancers tested here. The proven ability of this platform to accurately quantify nucleic acid traces, to validate potential miRNA biomarkers, and its potential adaptability to future solid-state nanopore platforms are unprecedented.

### Yenos Test

It has been discovered that a portable nanopore device from Oxford Nanopore Technologies (ONT) can be repurposed to detect a DNA/RNA polynucleotide (target) in a complex mixture by conducting voltage-driven ion-channel measurements. The detection and quantitation of the target was enabled by the use of a unique complementary probe of the present disclosure. Using a validated labeling technology, probes are tagged with a bulky Osmium tag (Osmium tetroxide 2,2′-bipyridine), in a way that preserves strong hybridization between probe and target. Untagged oligos traverse the nanopore relatively quickly compared to the device's acquisition rate and exhibit count of events comparable to the baseline. Counts can be reported, for example, by a publicly available software, OsBp_detect (Kanavarioti & Kang, 2020). Due to the presence of the bulky Osmium tag, osmium-tagged probes traverse more slowly, producing multiple counts over the baseline, and can even be detected in the single digit femtomolar (fM) range. In the presence of the target, however, the probe is “silenced”. Silencing is attributed to a double-stranded complex that for practical purposes of this disclosure are considered not to traverse the nanopore under the applied conditions, as the size of the protein nanopore of the MinION only fits single-stranded nucleic acid, such as the osmylated-probe of this disclosure. Thus, the disclosed ready-to-use platform can be tailored as a diagnostic test to meet the requirements, for example, of point-of-care circulating tumor DNA (ctDNA), cell free DNA (cfDNA) fragmented RNA, and microRNA (miRNA) detection and quantitation in body fluids.

### Materials and Methods

Human samples: Human serum from the USA isolated via sterile filtration from male AB-clotted whole blood (H6914, 1^{st} lot SLCH8785, 2^{nd} lot SLCJ3635; data reported earlier; 3^{rd} lot SLCL6534 and 4^{th} lot SLCN9213; data reported here in Table 2) were purchased from Sigma-Aldrich over a period of 3 years. Serum samples purchased from Discovery Life Sciences (DLS, Huntsville, AL) and Tissue for Research (Accio Biobank online, Suffolk, UK) were collected from informed consented individuals under the IRB/EC protocol. Selection of these samples from a large depository included both male and female donors, if applicable, and one each from African American, Hispanic, or White ethnicity. Samples were collected from newly diagnosed, naïve, pretreatment patients. The demographic information of the cancer patients who provided their specimens is listed in Table 5. The project to include urine samples collected from consented healthy subjects was reviewed by the Advarra Investigational Review Board (IRB). The protocol and the consent form were reviewed, modified, and approved by the Advarra IRB on Nov. 15, 2023. Protocol: Yenos Analytical LLC-02. Quantification of selected microRNAs in the urine of healthy individuals (Pro00074065). Urine sample donors reviewed and signed the informed consent form. They were then sent a kit/insulated box, cold bricks, and instructions to collect their biospecimen at home, freeze it and ship it back to the Yenos Analytical Laboratory facilities. The biospecimens were shipped overnight and received cold at Yenos' facility. The healthy urine donors were 22 to 72 years old, female, or male and of different ethnicities. For the isolation of RNA from serum, we used the Monarch T2010S Kit (1^{st} lot 10075450, 2^{nd} lot 10141109 reported earlier, and 3^{rd} lot 10144556 used here). For the isolation of total RNA from urine, two kits were used: No. 29000 was used for 1 mL urine samples, and the slurry kit No. 29600 was used for 5 to 10 mL urine samples. All kits were used according to the manufacturer's instructions.

TABLE 5
Demographics of cancer patients whose samples are listed in Table 3 and Table 4.

| biobank | ID | age | gender | cancer | T stage | N Stage | specimen |
|---|---|---|---|---|---|---|---|
| Tissue for | 101499 | 59 | F | breast | — | — | serum |
| Research | | | | | | | matched |
| | SR23 6016 | 55 | F | | pT1b | pN0 | urine |
| | SR23 6017 | 66 | F | ″ | pT1b | ″ | ″ |
| | SR23 6018 | 54 | F | ″ | pT1a | ″ | ″ |
| | SR23 6022 | 74 | M | prostate | pT2 | ″ | ″ |
| | SR23 6023 | 66 | M | ″ | ″ | ″ | ″ |
| | SR23 6028 | 54 | M | ″ | ″ | ″ | ″ |
| | SR23 6033 | 66 | F | pancreatic | ″ | ″ | ″ |
| | SR16 690 | 55 | M | ″ | pT2 | ″ | serum |
| | SR17 248 | 55 | M | ″ | pT1 | ″ | ″ |
| Discovery | CAN4 | 66 | M | prostate | newly diagnosed, | | serum |
| Life | CAN6 | 56 | M | ″ | pretreatment | | ″ |
| Sciences | CAN7 | 52 | F | breast | | | ″ |
| | CAN9 | 58 | F | ″ | | | ″ |

Oligos, Probes, and other Reagents: The only ONT kits used for the experiments reported here were a Flow Cell Priming Kit XL (EXP-FLP002-XL), ONT flush buffer or ONT buffer. ONT buffer is proprietary, provides the necessary electrolytes and must represent more than 80% of the approximate 80 μL sample volume. Custom-made DNA oligos and 2′-OMe-oligos synthesized at the 0.2 μM scale and purified by HPLC were purchased from Integrated DNA Technologies (IDT) and Millipore/Sigma-Aldrich, respectively. Oligos (sequences in Table 6) were diluted with Ambion nuclease-free water, not treated with DEPC, typically to 100 or 200 μM stock solutions, and stored at −20° C. The oligo purity was confirmed by in-house HPLC analysis to be >85% [Kanavarioti (2019)]. Following osmium tagging (osmylation, see below), in-house HPLC analysis was used to determine the probe content, extent of osmylation, and efficiency of probe/target hybridization. Osmylated oligos were manufactured in house based on published methods at a concentration of approximately 30 μM. LoBind Eppendorf test tubes (1.5 mL) were used for serial 5/1000 or 10/1000 dilutions to yield probes at 15 to 30 fM concentrations. Mixtures of probe with isolated RNA were prepared in 0.5 mL RNase-, DNase-free, sterile test tubes and stored at −20° C. overnight.

Osmylation of nucleic acids using a 1:1 mixture of OsO₄ and 2,2′-bipyridine, abbreviated OsBp, was discovered 60 years ago [Chang et al. (1977)], used extensively [Palecek (1992); Reske et al. (2009); Debnath & Okamoto (2018)], and optimized by us [Kanavarioti et al. (2012); Kanavarioti (2016)]. The detailed protocols for the synthesis, purification and quality control assays have been described previously [Kang et al. (2020); Kanavarioti (2022)]. OsO₄ is a hazardous material, and care needs to be taken before its use, storage, and disposal [MSDS, UCLA]. Osmylation reactions require a 20-fold excess of OsBp over the reactive pyrimidine in monomer equivalents to ensure pseudo-first-order kinetics and yield preferential labeling of thymidines (T) over the other pyrimidines. Quenching of the osmylation reaction occurs upon purification. Purification from excess OsBp (twice) was performed with spin columns (TC-100 FC from TrimGen Corporation) for 4 min at 5,000 rpm. The flow-through solution is the probe, which is chemically stable and may be stored at −20° C. for two years.

The development, optimization, and validation of probes for enhanced MinION detection were previously reported [Kanavarioti (2022)]. The optimized probe comprises a sequence complementary to its target but extended at one end with 4 to 5 adjacent T residues and flanked by up to 5 adenosines (A) at either end (Table 6). The As at either end facilitated probe entry into the nanopores. The adjacent Ts were tagged with an osmium label for quantitative detection. Within the probe sequence complementary to the target, Ts is replaced by uridine (U), 2′-OMe-U or dU to minimize OsBp labeling because the osmylation kinetics of U and cytosine (C) are substantially slower than that of T [Kanavarioti et al. (2012)]. HPLC analysis yields the probe concentration (content) using the intact oligo as a standard because the absorbance of the probe at 260 nm is practically the same as that of the precursor intact oligo [Kanavarioti et al. (2012)]. HPLC analysis provides evidence for quantitative depletion of the OsBp reagent. Alternatively, a NanoDrop spectrophotometer can be used to determine the content and extent of osmylation.

TABLE 6
Sequence and characterization of the probes (see sequence listing)

| ID: DNA oligo | | | |
|---|---|---|---|
| sequence used | | Conc. ¹ | # OsBp |
| for probe | In Sequence mU is 2′-OMeU and dU is 2′-deoxyU | fM | ave ² |
| TC(10) | TCTCTCTCTCTCTCTCTCTC | — | 0, not a |
| SEQ ID NO: 1 | | | probe |
| T8(9 OsBp), RNA | (AG)₄C₂(AG)₄C₂(AG)₃CCUUC | — | 8 |
| SEQ ID NO: 2 | | | |
| Probe 375T5 | (A)₅dUCACGCGAGCCGAACGAACAAAC(T)₅C(A)₅ | 42.0 | 5.1 |
| SEQ ID NO: 3 | | | |
| Probe m21T5 | (A)₅mUCAACAmUCAGmUCmUGAmUAAGCmUA(T)₅C(A)₆ | 27.1 | 4.4 |
| SEQ ID NO: 4 | | | |
| Probe m141T5 | (A)₄CCAmUC(mU)₃ACCAGACAGmUG(mU)₂A(T)₅(A)₅ | 33.5 | 4.7 |
| SEQ ID NO: 5 | | | |
| Probe 15bT5 | (A)₆dUGdUAAACCAdUGAdUGdUGCdUGCdUAT₅A₆ | 35.0 | 5.9 |
| SEQ ID NO: 6 | | | |
| Probe let7bT5 | (A)₆CCACACAACCmUACmUACCmUCA(T)₅(A)₅ | 30.0 | 5.5 |
| SEQ ID NO: 7 | | | |

¹ Concentration of the probe solution (fM) used for the nanopore experiments. It is obtained by 5/1000 or 10/1000 dilutions from the stock solution (mM) of the probe prepared by osmylation (T-OsBp)₅ of the oligo and characterized by HPLC in house.
² The average number of osmium label moieties on the probe (extent of osmylation) was measured using the equation: Absorbance at 312 nm/Absorbance at 272 nm or R(312/272) = 2x(No of osmylated pyrimidines/total nt)[Kanavarioti, 2022]. R is the ratio of the corresponding HPLC peaks, regardless of shape (sharp or broad). An extra osmium tag is conjugated with a C or U base within the sequence. A single internal tag does not prevent hybridization, as shown by nanopore experiments.

Single-molecule ion-channel conductance experiments on the MinION (MinION Mk1B platform): One must register with ONT and download the software MinKNOW to a computer/laptop (specs are provided by ONT). All the functions necessary to test the hardware and flow cells and run the experiments were performed via MinKNOW software. The sample is loaded on the flow cell that fits within the MinION device. The experiment was run under “start sequencing” mode. A direct RNA sequencing kit (SQK-RNA002) was used for the experiment. The flow cell type FLO-MIN106 was selected, and the run length (45 min) and bias voltage (−180 mV) were selected; basecalling was disabled, and the output bulk file Raw (1-512) was generated. The output location is/Library/MinKNOW/data/, and the output format is fast-5. All the experiments reported here were run for 45 min at −180 mV. Work under different conditions was performed earlier. Analysis of the fast-5 file was performed using the OsBp_detect algorithm [Kanavarioti & Kang, github]. The number of events per channel from the OsBp_detect analysis was compared to the actual i-t trace of the specific channel by MATLAB visualization, and this algorithm, 2^{nd} revision, was repeatedly validated. OsBp_detect can be used only with a 2017 or an earlier version of the MacBook Pro loaded with macOS 10.14 Mojave. Future work will include the adaptation of OsBp_detect to newer operating systems. While alternative parameters were explored, all the experiments reported here were analyzed using the following threshold parameters: (i) event duration (in tps): 4-1200 (1.3-400 ms), (ii) lowest Iᵣ/Iₒ<0.55, and (iii) all Iᵣ/Iₒ<0.6, channels 1-512.

Data analysis: A state-of-the-art laptop/computer requires approximately 5 min for the OsBp_detect analysis, produces a file in the tsv format, opens via Microsoft Excel and saves as such. In the Excel spreadsheet, the algorithm-selected events (Iᵣ/Iₒ data) are grouped in the form of a histogram with 0.05 bins, from 0.05 to 0.55, and plotted (FIG. 6 and FIG. 7). These events are added together and identified as Total Events. Typical histograms exhibit two maxima (Iᵣ/Iₒ)ₘₐₓ, an early one at Iᵣ/Iₒ=0.15 and a late one at Iᵣ/Iₒ=0.30. These maxima may vary by ±0.05 units depending on the flow cell age. The events under late (Iᵣ/Iₒ)ₘₐₓ and under early (Iᵣ/Iₒ)ₘₐₓ are noted, and their ratio ((Iᵣ/Iₒ)ₘₐₓlate (0.3)/early (0.15)) is calculated. These values (total events and ratio) represent the criteria by which an experiment is judged as detection or silencing compared to the buffer control. Fewer total events than in the control group suggested silencing, while more total events suggested detection. A decreasing ratio indicates detection, and an increasing ratio indicates silencing. This assignment is consistent with an increased number of events due to the presence of the probe, which traverses with (Iᵣ/Iₒ)ₘₐₓ˜0.15, while intact RNA and background noise traverse mostly with (Iᵣ/Iₒ)ₘₐₓ˜0.35. Each sample was run twice, and every run was compared to the buffer/control separately. Because flow cells lose active pores during every experiment, the total number of events decreases during every experiment. Fewer nanopores reduce the effect of the events due to the free probe. In contrast, fewer nanopores enhance the effect of fewer events due to the absence of the probe. The presence of the hybrid has an additional effect on reducing the number of events. This is because the hybrids are driven toward the nanopores, cannot go through, and are pushed back by the alternating voltage of the platform; however, they remain in proximity and prevent other molecules from traversing the nanopores.

Each figure contains a test, i.e., a set of 3 experiments with buffer (dotted line, open circles), followed by the 1^{st} run of the sample which is the mixture of RNA with the probe (dashed line, triangles) and followed by a 2^{nd} run of the same sample (solid line, filled circles). All 3 experiments were conducted at −180 mV for 45 min. Analysis of the events by OsBp_detect typically yields two maxima, one early Ir/Io=0.15 and a late Ir/Io=0.3. As seen the buffer alone exhibits events at both maxima, but the Yenos probes traverse only at Ir/Io=0.15. Therefore, the presence of free probe is consistent with an increase at the early Ir/Io and/or a decrease at the late Ir/Io because there is a steady decrease of events due to the inactivation of the nanopores. Silencing experiments often exhibit a markedly reduced number of events due to nanopore “shielding” as discussed in the text, while detection experiments (top) exhibit comparable counts but a reversed distribution with relatively more events at the early (Iᵣ/Iₒ)ₘₐₓ=0.15 and fewer events at the late (Iᵣ/Iₒ)ₘₐₓ=0.30. For a specific experiment with an x μL probe and y μL sample RNA, one calculates probe molecules P=x mL (probe concentration in fM)×600. If the experiment involved detection, then P>target miRNA molecules within the y μL aliquot. If the experiment involved silencing, then P<target miRNA molecules in y μL. It follows that the number of miRNA molecules per 1 μL of isolated RNA sample < or >P/y, depending on the experimental outcome.

Most, if not all, analytical assays make a single measurement, for example, a UV-vis spectrophotometer. For a given sample, there will be one measurement, such as Absorbance=10 mAU. Depending on the sample, the accuracy of the measurement varies. If the sample is very dilute and close to the detectability limit of the detector, then the accuracy is low, could be as low as +/−40%. One can determine the accuracy by measuring the same sample multiple times and get an RSD of about 40%. Then one knows that for any sample at this level of concentration, the accuracy is going to be close to 40%. Practically speaking, any measurement which is close to the noise of the instrument, is going to be less accurate than another measurement which is much further than the noise. In the UV-vis, this translates to concentration.

The methods disclosed herein are fundamentally different. There is no way to make one measurement and obtain a miRNA copy number. For example, take a sample with 1000 miR-21 copies per 1 μL. A set of experiments is prepared (Samples A through E) with 1 μL amount from the sample and different amounts of the probe for miR-21.
Sample A: 1000 miR-21 copies and 2000 probe miR-21 copies
Sample B: 1000 miR-21 copies and 1600 probe miR-21 copies
Sample C: 1000 miR-21 copies and 1200 probe miR-21 copies
Sample D: 1000 miR-21 copies and 800 probe miR-21 copies
Sample E: 1000 miR-21 copies and 400 probe miR-21 copies

Samples A, B, and C will be characterized by the test as “detection” experiments because the probe copies are more than the target. Samples D and E will be characterized as “silencing” experiments, because the probe copies are less than the target.

To determine the number of miR-21 copies, Samples C and D are selected because they lie closest to each other, and because one is a detection and the other is a silencing experiment. The average is calculated to determine that miR-21 measures 1000 copies in the sample. This measurement is at +/−20% accuracy (from 1200+800=2000/2=1000+/−200). For example, if the selected experiments were 1300 (detection) and 900 (silencing), then the determination would yield an average of 1100, again at a +/−20% accuracy.

Where the probe test described above exhibits the same +/−20% accuracy across all concentrations, this is the complete opposite from the UV-vis spectrophotometer case discussed above. This doesn't mean that there is no noise/background to consider in the tests described here. It means, however, that because it is a comparison of two entities, and because one of the two entities (the probe) is known, the experiments can be protocol-defined to be above the noise. Theoretically, one reaches the same conclusion if one tests 1000 miR-21 copies or 100 miR-21 copies. As the “noise” in the system is known, the optimum range of copies is selected, so that the noise is never an issue.

In another example, a user wants to do less experiments and selects only 2000, 1300, and 600 probe miR-21 copies. If the miR-21 copy number is 1000, one will obtain two detection results and one silencing. The average will be 950+/−350 (i.e., (1300+600)/2)). This result will have an accuracy at about +/−35%, If the chosen experiments had been, for example, 2400, 1600, and 800 probe miR-21 copies, the average would have been determined to be miR-21=1200+/−400, at an accuracy of 33%. As Table 1A and Table 1B show, if the x-fold between disease and control group is larger, then the accuracy can be less, which typically translates to fewer experiments.

The breadth and scope of the present disclosure should not be limited by any of the above-described exemplary embodiments but should be defined only in accordance with the following claims and their equivalents.

### REFERENCES

Alahdal et al. (2023): Alahdal, M., Perera, R. A., Moschovas, M. C., Patel, V., & Perera, R. J. (2023). Current advances of liquid biopsies in prostate cancer: Molecular biomarkers. Molecular therapy oncolytics, 30, 27-38. ttps://doi.org/10.1016/j.omto.2023.07.004
Alles et al. (2019): Alles, J. et al. An estimate of the total number of true human miRNAs. Nucleic Acids Research, 47, 3353-3364 (2019). https://doi.org/10.1093/nar/gkz097
Ambros (2001): Ambros, V. MicroRNAs: tiny regulators with great potential. Cell, 107, 823-826, (2001). doi: 10.1016/S0092-8674 (01) 00616-X.
Armakolas et al. (2023): Armakolas, A., Kotsari, M., & Koskinas, J. (2023). Liquid Biopsies, Novel Approaches and Future Directions. Cancers, 15(5), 1579. https://doi.org/10.3390/cancers 15051579
Ban et al. (2023): Ban, E., Lim, H. J., Kwon, H., & Song, E. J. (2023). Practical magnetic bead-based capillary electrophoresis with laser-induced fluorescence for detecting endogenous miRNA in plasma. Heliyon, 9(12), e22809. https://doi.org/10.1016/j.heliyon.2023.e22809
Bartel (2004): Bartel, D. P. MicroRNAs: genomics, biogenesis, mechanism, and function. Cell, 116, 281-297 (2004). doi: 10.1016/S0092-8674 (04) 00045-5.
Beer (2020): Beer T. M. (2020). Novel blood-based early cancer detection: diagnostics in development. The American journal of managed care, 26(14 Suppl), S292-S299. https://doi.org/10.37765/ajmc.2020.88533
Benke et al. (2023): Benke, M., Zeöld, A., Kittel, Á., Khamari, D., Hritz, I., Horváth, M., Keczer, B., Borka, K., Szücs, Á., & Wiener, Z. (2023). MiR-200b categorizes patients into pancreas cystic lesion subgroups with different malignant potential. Sci Rep, 13(1), 19820. https://doi.org/10.1038/s41598-023-47129-1
Borbiev et al. (2023): Borbiev, T., Kohaar, I., & Petrovics, G. Borbiev, T., Kohaar, I., & Petrovics, G. (2023). Clinical Biofluid Assays for Prostate Cancer. Cancers, 16(1), 165. https://doi.org/10.3390/cancers16010165.
Butler et al. (2007): Butler, T. Z., Gundlach, J. H. & Troll, M. Ionic current blockades from DNA and RNA molecules in the alpha-hemolysin nanopore. Biophys J. 93, 3229-3240 (2007). https://doi.org/10.1529/biophysj. 107.107003
Cai et al. (2021): Cai, S., Pataillot-Meakin, T., Shibakawa, A., Ren, R., Bevan, C. L., Ladame, S., Ivanov, A. P., & Edel, J. B. (2021). Single-molecule amplification-free multiplexed detection of circulating microRNA cancer biomarkers from serum. Nature communications, 12(1), 3515. https://doi.org/10.1038/s41467-021-23497-y
Chang et al. (1977): Chang, C. H., Beer, M. & Marzilli, L. G. Osmium-labeled polynucleotides. The reaction of osmium tetroxide with deoxyribonucleic acids and synthetic polynucleotides in the presence of tertiary nitrogen donor ligands. Biochem. 16, 33-38 (1977). https://doi.org/10.1021/bi00620a006
Chomczynski et al. (2016): Chomczynski P, Wilfinger W W, Eghbalnia H R, Kennedy A, Rymaszewski M, et al. (2016) Inter-Individual Differences in RNA Levels in Human Peripheral Blood. PLOS ONE 11 (2): e0148260. https://doi.org/10.1371/journal.pone.0148260
Cohen et al. (2018): Cohen, J. D., Li, L., Wang, Y., Thoburn, C., Afsari, B., Danilova, L., Douville, C., Javed, A. A., Wong, F., Mattox, A., Hruban, R. H., Wolfgang, C. L., Goggins, M. G., Dal Molin, M., Wang, T. L., Roden, R., Klein, A. P., Ptak, J., Dobbyn, L., Schaefer, J., . . . Papadopoulos, N. (2018). Detection and localization of surgically resectable cancers with a multianalyte blood test. Science (New York, N.Y.), 359 (6378), 926-930. https://doi.org/10.1126/science.aar3247
Connal et al. (2023): Connal, S., Cameron, J. M., Sala, A., Brennan, P. M., Palmer, D. S., Palmer, J. D., Perlow, H., & Baker, M. J. (2023). Liquid biopsies: the future of cancer early detection. Journal of translational medicine, 21(1), 118. https://doi.org/10.1186/s12967-023-03960-8
Debnath & Okamoto (2018): Debnath, T. K. & Okamoto, A. Osmium tag for posttranscriptionally modified RNA. ChemBioChem, 19, 1653-1656 (2018). https://doi.org/10.1002/cbic.201800274
Ding & Kanavarioti (2016): Ding, Y. & Kanavarioti, A. Single pyrimidine discrimination during voltage-driven translocation of osmylated oligodeoxynucleotides via the α-hemolysin nanopore. Beilstein J Nanotechnol. 7, 91-101 (2016). https://doi.org/10.3762/bjnano.7.11
Duffy & Crown (2023): Duffy, M. J., & Crown, J. (2023). Circulating tumor DNA (ctDNA): can it be used as a pancancer early detection test? Critical reviews in clinical laboratory sciences, 1-13. Advance online publication. https://doi.org/10.1080/10408363.2023.2275150
Filella & Foj (2017): Filella, X., & Foj, L. (2017). miRNAs as novel biomarkers in the management of prostate cancer. Clinical chemistry and laboratory medicine, 55(5), 715-736. https://doi.org/10.1515/cclm-2015-1073
Gahlawat et al. (2023): Gahlawat, A. W., Witte, T., Sinn, P., & Schott, S. (2023). Circulating cf-miRNA as a more appropriate surrogate liquid biopsy marker than cfDNA for ovarian cancer. Scientific reports, 13 (1), 5503. https://doi.org/10.1038/s41598-023-32243-x
Gao et al. (2016): Gao, Y., Feng, B., Han, S., Zhang, K., Chen, J., Li, C., Wang, R., & Chen, L. (2016). The Roles of MicroRNA-141 in Human Cancers: From Diagnosis to Treatment. Cellular physiology and biochemistry: international journal of experimental cellular physiology, biochemistry, and pharmacology, 38(2), 427-448. https://doi.org/10.1159/000438641
Godoy et al. (2019): Godoy P M, Barczak A J, DeHoff P, et al. Comparison of Reproducibility, Accuracy, Sensitivity, and Specificity of miRNA Quantification Platforms. Cell Rep. 29, 4212-4222.e5 (2019). doi:10.1016/j.celrep.2019.11.078
Haque et al. (2013): Haque, F., Li, J., Wu, H. C., Liang, X. J. & Guo, P. Solid-state and biological nanopore for real-time sensing of single chemical and sequencing of DNA. Nano Today, 8, 56-74 (2013). https://doi.org/10.1016/j.nantod.2012.12.008
Henley et al. (2015): Henley, R. Y., Vazquez-Pagan, A. G., Johnson, M., Kanavarioti, A. & Wanunu, M. Osmium-based on pyrimidine contrast tags for enhanced nanopore-based DNA base discrimination. PLOS One, 10(12), e0142155, (2015). https://doi.org/10.1371/journal.pone.0142155
Hindson et al. (2013): Hindson, C. M., Chevillet, J. R., Briggs, H. A., Gallichotte, E. N., Ruf, I. K., Hindson, B. J., Vessella, R. L., & Tewari, M. (2013). Absolute quantification by droplet digital PCR versus analog real-time PCR. Nature methods, 10(10), 1003-1005. https://doi.org/10.1038/nmeth.2633
Jenike & Halushka (2021): Jenike, A. E., & Halushka, M. K. miR-21: a nonspecific biomarker of all maladies. Review. Biomarker research, 9, 18 (2021). https://doi.org/10.1186/s40364-021-00272-1
Jet et al. (2021): Jet, T., Gines, G., Rondelez, Y., & Taly, V., (2021). Advances in multiplexed techniques for the detection and quantification of microRNAs. Chemical Society reviews, 50(6), 4141-4161. https://doi.org/10.1039/docs00609b
Kanavarioti (2019): Kanavarioti, A. HPLC methods for purity evaluation of man-made single-stranded RNAs. Sci Rep. 9, 1019 (2019). https://doi.org/10.1038/s41598-018-37642-z Kanavarioti et al. (2012): Kanavarioti, A. et al. Capillary electrophoretic separation-based approach to determine the labeling kinetics of oligodeoxynucleotides. Electrophoresis, 33, 3529-3543 (2012). https://doi.org/10.1002/elps.201200214
Kanavarioti (2016): Kanavarioti, A. False positives and false negatives measure less than 0.001% in labeling ssDNA with osmium tetroxide 2,2′-bipyridine. Beilstein J Nanotechnol, 7, 1434-1446 (2016). https://doi.org/10.3762/bjnano.7.135
Kanavarioti & Kang (2020) in github.com: Kanavarioti, A. & Kang, A. S. W. See RNA (OsBp) event detection Python package in a public repository: https://github.com/kangaroo96/osbp_detect and for step-by-step installation instructions see here: https://github.com/kangaroo96/osbp_detect/blob/master/instructions.md. The reasoning behind the development of the algorithm is detailed in the Experimental Section of Reference Kang et al. (2020).
Kanavarioti (2022): Kanavarioti, A. Femtomolar-level PCR-free quantification of microRNA cancer biomarkers in serum, posted in BioRxiv, doi: https://doi.org/10.1101/2022.12.30.522268
Kang et al. (2020): Kang, A. S. W., Bernasconi, J. G., Jack, W., & Kanavarioti, A. (2020). Ready-to-use nanopore platform for the detection of any DNA/RNA oligo at attomole range using an Osmium tagged complementary probe. Scientific reports, 10 (1), 19790. https://doi.org/10.1038/s41598-020-76667-1
Kane et al. (2022): Kane, L. E., Mellotte, G. S., Mylod, E., O'Brien, R. M., O'Connell, F., Buckley, C. E., Arlow, J., Nguyen, K., Mockler, D., Meade, A. D., Ryan, B. M., & Maher, S. G. (2022). Diagnostic Accuracy of Blood-based Biomarkers for Pancreatic Cancer: A Systematic Review and Meta-analysis. Cancer research communications, 2(10), 1229-1243. https://doi.org/10.1158/2767-9764.CRC-22-0190
Kasianowicz et al. (1996): Kasianowicz, J. J., Brandin, E., Branton, D. & Deamer, D. W. Characterization of individual polynucleotide molecules using a membrane channel. Proc Natl Acad Sci USA, 93, 13770-13773 (1996). doi: 10.1073/pnas.93.24.13770.
Koch et al. (2023): Koch, C., Reilly O'Donnell, B., Gutierrez, R., Lucarelli, C., Ng, F. S., Gorelik, J., Ivanov, A. P., & Edel, J. B. (2023). Nanopore sequencing of DNA-barcoded probes for highly multiplexed detection of microRNA, proteins and small biomarkers. Nature nanotechnology, 18(12), 1483-1491. https://doi.org/10.1038/s41565-023-01479-z
Komen-mammograms: https://www.Komen.org, Accuracy of mammograms
Kozomara et al. (2019): Kozomara, A., Birgaoanu, M., & Griffiths-Jones, S. (2019). miRBase: from microRNA sequences to function. Nucleic acids research, 47(D1), D155-D162. https://doi.org/10.1093/nar/gkyl141
Maglia et al. (2010): Maglia, G., Heron, A. J., Stoddart, D., Japrung, D. & Bayley, H. Analysis of single nucleic acid molecules with protein nanopores. Methods Enzymol. 475, 591-623 (2010). https://doi.org/10.1016/S0076-6879 (10) 75022-9
Maguire & Guan (2022): Maguire, S., & Guan, S. (2022). Rolling circle reverse transcription enables high fidelity nanopore sequencing of small RNA. PloS one, 17(10), e0275471. https://doi.org/10.1371/journal.pone.0275471
Mall et al. (2013): Mall, C., Rocke, D. M., Durbin-Johnson, B., & Weiss, R. H. Stability of miRNA in human urine supports its biomarker potential. Biomarkers in Medicine 7, 623-631 (2013). https://doi.org/10.2217/bmm. 13.44
Mestdagh et al. (2014): Mestdagh, P., Hartmann, N., Baeriswyl, L., Andreasen, D., Bernard, N., Chen, C., Cheo, D., D'Andrade, P., DeMayo, M., Dennis, L., Derveaux, S., Feng, Y., Fulmer-Smentek, S., Gerstmayer, B., Gouffon, J., Grimley, C., Lader, E., Lee, K. Y., Luo, S., Mouritzen, P., . . . Vandesompele, J. (2014). Evaluation of quantitative miRNA expression platforms in the microRNA quality control (miRQC) study. Nature methods, 11(8), 809-815. https://doi.org/10.1038/nmeth.3014
Mitchell et al. (2008): Mitchell, P. et al. Circulating microRNAs as stable blood-based markers for cancer detection. Proc Natl Acad Sci USA, 105, 10513-10518 (2008).
doi: 10.1073/pnas.0804549105.
MSDS UCLA: MSDS and Information is obtained from the following link to UCLA Chemistry Depart., see https://www.chemistry.ucla.edu/sites/default/files/safety/sop/SOP_Osmium_Tetroxide.pdf
Nicholson et al. (2023): Nicholson, B. D., Oke, J., Virdee, P. S., Harris, D. A., O'Doherty, C., Park, J. E., Hamady, Z., Sehgal, V., Millar, A., Medley, L., Tonner, S., Vargova, M., Engonidou, L., Riahi, K., Luan, Y., Hiom, S., Kumar, H., Nandani, H., Kurtzman, K. N., Yu, L. M., . . . Middleton, M. R. (2023). Multicancer early detection test in symptomatic patients referred for cancer investigation in England and Wales (SYMPLIFY): a large-scale, observational cohort study. The Lancet. Oncology, 24(7), 733-743. https://doi.org/10.1016/S1470-2045 (23) 00277-2; and https://grail.com/clinical-studies/
NHSE, Prostate: National Health Service England, Prostate, https://www.nhs.uk/conditions/prostate-cancer/should-i-have-psa-test/NIH/NCI:
NIH, National Cancer Institute, Age and Cancer Risk.
Palecek (1992): Palecek, E. Probing DNA structure with osmium tetroxide complexes in vitro. Methods in Enzymology, 212, 139-155, (1992). https://www.ncbi.nlm.nih.gov/pubmed/1518446
Porzycki et al. (2018): Porzycki, P., Ciszkowicz, E., Semik, M., Tyrka, M. Combination of three miRNA (miR-141, miR-21, and miR-375) as potential diagnostic tool for prostate cancer recognition. Int Urol Nephrol. 50, 1619-1626 (2018). doi:10.1007/s11255-018-1938-2
Quint et al. (2024): Quint, I., Simantzik, J., Kaiser, L., Laufer, S., Csuk, R., Smith, D., Kohl, M., & Deigner, H. P. (2024). Ready-to-use nanopore platform for label-free small molecule quantification: Ethanolamine as first example. Nanomedicine: nanotechnology, biology, and medicine, 55, 102724. https://doi.org/10.1016/j.nano.2023.102724
Reske et al. (2009): Reske, T., Surkus, A-E., Duwensee, H. & Flechsig, G.-U. Kinetics of the labeling reactions of thymine, cytosine and uracil with osmium tetroxide bipyridine. Microchimica Acta, 166, 197-201 (2009). https://doi.org/10.1007/s00604-009-0195-6
Robinson et al. (2018): Robinson, S., Follo, M., Haenel, D., Mauler, M., Stallmann, D., Heger, L. A., Helbing, T., Duerschmied, D., Peter, K., Bode, C., Ahrens, I., & Hortmann, M. (2018). Chip-based digital PCR as a novel detection method for quantifying microRNAs in acute myocardial infarction patients. Acta pharmacologica Sinica, 39(7), 1217-1227. https://doi.org/10.1038/aps.2017.136
Sequeira et al. (2023): Sequeira, J. P., Barros-Silva, D., Ferreira-Torre, P., Salta, S., Braga, I., Carvalho, J., Freitas, R., Henrique, R., & Jerónimo, C. (2023). OncoUroMiR: Circulating miRNAs for Detection and Discrimination of the Main Urological Cancers Using a ddPCR-Based Approach. International journal of molecular sciences, 24(18), 13890. https://doi.org/10.3390/ijms241813890
Seyhan (2023): Seyhan A. A. (2023). Circulating microRNAs as Potential Biomarkers in Pancreatic Cancer-Advances and Challenges. International journal of molecular sciences, 24(17), 13340. https://doi.org/10.3390/ijms241713340
Siegel et al. (2023): Siegel R L, Miller K D, Wagle N S, Jemal A. Cancer statistics, 2023. CA Cancer J Clin. 2023; 73(1): 17-48. doi: 10.3322/caac.21763
Smolarz et al. (2022): Smolarz, B., Durczyński, A., Romanowicz, H., Szyllo, K., & Hogendorf, P. (2022). miRNAs in Cancer (Review of Literature). International journal of molecular sciences, 23 (5), 2805. https://doi.org/10.3390/ijms23052805
Stosic et al. (2023): Stosic, K., Senar, O. A., Tarfouss, J., Bouchart, C., Navez, J., Van Laethem, J. L., & Arsenijevic, T. (2023). A Comprehensive Review of the Potential Role of Liquid Biopsy as a Diagnostic, Prognostic, and Predictive Biomarker in Pancreatic Ductal Adenocarcinoma. Cells, 13(1), 3. https://doi.org/10.3390/cells13010003
Sultan & Kanavarioti (2019): Sultan M. & Kanavarioti, A. Nanopore device-based fingerprinting of RNA oligos and microRNAs enhanced with an osmium tag. Sci Rep. 9, 14180 (2019). https://doi.org/10.1038/s41598-019-50459-8
Valihrach et al. (2020): Valihrach, L., Androvic, P. & Kubista, M. Circulating miRNA analysis for cancer diagnostics and therapy. Molecular Aspects of Medicine 72, 100825 (2020). https://doi.org/10.1016/j.mam.2019.10.002
WHO Cancer: World Health Organization Cancer, https://www.who.int/news-room/fact-sheets/detail/cancer
WHN Mammograms: Women's Health Network Mammograms, https://www.womenshealthnetwork.com/breast-health/mammograms/
Xi et al. (2016): Xi, D. et al. Nanopore-based selective discrimination of microRNAs with single-nucleotide difference using locked nucleic acid-modified probes. Anal. Chem. 88, 10540-10546 (2016). https://doi.org/10.1021/acs.analchem.6b02620
Zahid et al. (2016): Zahid, O. K., Wang, F., Ruzicka, J. A., Taylor, E. W. & Hall, A. R. Sequence-specific recognition of microRNAs and other short nucleic acids with solid-state nanopores. Nano Lett, 16, 2033-2039 (2016). https: doi.org/10.1021/acs.nanolett.6b00001
Zografos et al. (2019): Zografos, E., Zagouri, F., Kalapanida, D. et al. Prognostic role of microRNAs in breast cancer: A systematic review. Oncotarget. 10, 7156-7178 (2019). doi:10.18632/oncotarget.27327.

1. A method of validating a miRNA as a biomarker indicative of a disease, the method comprising:
(a) measuring a control miRNA copy number (H1) in a sample of isolated total RNA from a biospecimen of a control subject without the disease (isolated total RNA control sample) using an osmylated-probe that targets the miRNA and a nanopore detection method, wherein the accuracy of the control miRNA copy number measurement is protocol-defined,
optionally, wherein H1 is measured from a combined biospecimen of control subjects,
optionally, where the accuracy of the measurement is between +/−15% and +/−50%;
(b) repeating step (a) for one or more additional control samples (H2 to Hn), optionally, wherein n is at least 3, 4, 5, 6, 7, 8, 9, or 10;
(c) normalizing the miRNA copy numbers of the additional control samples H2 to Hn to the RNA content of the isolated total RNA control sample and selecting an average miRNA copy number from H1 to Hn as a control miRNA copy number (Hc),
optionally, wherein Hc is the average of the normalized H1 to Hn copy numbers with relative standard deviation (RSD) between 15% and 50%;
(d) measuring a disease miRNA copy number (D1), of the same miRNA measured in (a), in the isolated total RNA from a biospecimen of a disease subject confirmed to have the disease using the same osmylated probe used in (a) that targets the miRNA and the nanopore detection method, wherein the accuracy of the disease miRNA copy number measurement is protocol-defined,
optionally, wherein the accuracy of the disease miRNA copy number measurement is protocol-defined to be comparable to and/or match that of the control sample measurement;
(e) repeating step (d) for one or more additional disease samples (D2 to Dn), optionally, wherein Dn is at least 3, 4, 5, 6, 7, 8, 9, or 10;
(f) for overexpression, normalizing the miRNA copy numbers D1 to Dn of the disease samples to the RNA content of the isolated total RNA control sample and then dividing the disease sample normalized miRNA copy number values (D1(norm) to Dn(norm)) by the miRNA copy number of the control sample (Hc) to obtain a series of ratios of the normalized disease sample miRNA copy number to the control sample miRNA copy number (D1(norm)/Hc to Dn(norm)/Hc), or
for underexpression, normalizing the miRNA copy numbers D1 to Dn of the disease samples to the RNA content of the isolated total RNA control sample and then dividing the miRNA copy number of the control sample (Hc) by the disease sample normalized miRNA copy number values (D1(norm) to Dn(norm)) to obtain a series of ratios of the normalized disease sample miRNA copy number to the control sample miRNA copy number (Hc/D1(norm) to Hc/Dn(norm)); and
(g) making a determination whether the miRNA is a validated biomarker based on the ratios determined in (f).

2. The method of validating a miRNA as a biomarker indicative of a disease of claim 1,
wherein the accuracy of the measurement in steps (a), (b), (d) and/or (e) is protocol-defined to be between +/−15% and +/−40%; and/or
wherein the accuracy of the measurement in steps (a), (b), (d), and/or (e) is protocol-defined to be about +/−15%, +/−16%, +/−17%, +/−18%, +/−19%, +/−20%, +/−21%, +/−22%, +/−23%, +/−24%, +/−25%, +/−26%, +/−27%, +/−28%, +/−29%, or +/−30%.

3. The method of validating a miRNA as a biomarker indicative of a disease of claim 1, wherein the disease is a cancer indication, and the miRNA is validated as a biomarker of this cancer indication.

4. The method of validating a miRNA as a biomarker indicative of a disease of claim 1,
wherein the biospecimen is collected from the disease subject at an early stage (I or II) of the disease and/or ahead of treatment for the disease, and/or
wherein the biospecimen is collected before the subject is diagnosed with the disease and the subject later develops and/or is diagnosed with the disease.

5. The method of validating an overexpressed miRNA as a biomarker indicative of a disease of claim 1,
wherein if the RSD of the measurements H1 to Hn and D1 to Dn is less than or equal to 15% and all the ratios in the series D1(norm)/Hc to Dn(norm)/Hc are more than 1.35,
wherein if the RSD of the measurements H1 to Hn and D1 to Dn is less than or equal to 20% and all the ratios in the series D1(norm)/Hc to Dn(norm)/Hc are more than 1.5,
wherein if the RSD of the measurements H1 to Hn and D1 to Dn is less than or equal to 30% and all the ratios in the series D1(norm)/Hc to Dn(norm)/Hc are more than 1.85,
wherein if the RSD of the measurements H1 to Hn and D1 to Dn is less than or equal to 40% and all the ratios in the series D1(norm)/Hc to Dn(norm)/Hc are more than 2.3,
wherein if the RSD of the measurements H1 to Hn and D1 to Dn is less than or equal to 50% and all the ratios in the series D1(norm)/Hc to Dn(norm)/Hc are more than 3.0,
the miRNA is a validated overexpressed biomarker for the disease.

6. The method of validating an underexpressed miRNA as a biomarker indicative of a disease of claim 1,
wherein if the RSD of the measurements H1 to Hn and D1 to Dn is less than or equal to 15% and all the ratios in the series Hc/D1(norm) to Hc/Dn(norm) are more than 1.35, wherein if the RSD of the measurements H1 to Hn and D1 to Dn is less than or equal to 20% and all the ratios in the series Hc/D1(norm) to Hc/Dn(norm) are more than 1.5,
wherein if the RSD of the measurements H1 to Hn and D1 to Dn is less than or equal to 30% and all the ratios in the series Hc/D1(norm) to Hc/Dn(norm) are more than 1.85,
wherein if the RSD of the measurements H1 to Hn and D1 to Dn is less than or equal to 40% and all the ratios in the series Hc/D1(norm) to Hc/Dn(norm) are more than 2.3,
wherein if the RSD of the measurements H1 to Hn and D1 to Dn is less than or equal to 50% and all the ratios in the series Hc/D1(norm) to Hc/Dn(norm) are more than 3.0,
the miRNA is a validated underexpressed biomarker for the disease.

7. The method of validating a miRNA as a biomarker indicative of a disease of claim 1, wherein the method does not require nucleic acid amplification.

8. The method of validating a miRNA as a biomarker indicative of a disease of claim 1, wherein the control and disease subjects are an animal,
optionally, wherein the animal is a mammal,
further optionally, wherein the mammal is a human.

9. The method of validating a miRNA as a biomarker indicative of a disease of claim 8, wherein the animal is a mammal and the biospecimen is urine.

10. The method of validating a miRNA as a biomarker indicative of a disease of claim 1, wherein the control and disease subjects are a plant and the biospecimen is collected from leaf tissue, callus, stem tissue, root tissue, flowers, pollen, oil, sap, and/or seed.

11. A method of detecting a disease in a subject using as a biomarker a miRNA validated for the disease according to the method of claim 1,
optionally, wherein the disease is a cancer indication.

12. The method of detecting a disease of claim 11, wherein the subject is asymptomatic.

13. The method of detecting a disease of claim 11, wherein the validated miRNA biomarker is used to confirm a diagnosis of a disease.

14. The method of detecting a disease of claim 11, wherein the method comprises incubating a predetermined threshold amount of an osmylated-probe that binds the miRNA biomarker with a sample of a specified amount of RNA isolated from the subject, and using a nanopore detection method to determine whether there is an excess of probe in the incubated sample (detection) or an excess of miRNA biomarker in the incubated sample (silencing), wherein silencing is indicative of an elevated amount of the miRNA biomarker and that the subject has the disease.

15. The method of detecting a disease of claim 11, wherein the method comprises incubating a predetermined threshold amount of an osmylated-probe that binds the miRNA biomarker with a sample of a specified amount of RNA isolated from the subject, and using a nanopore detection method to determine whether there is an excess of probe in the incubated sample (detection) or an excess of miRNA biomarker in the incubated sample (silencing), wherein detection is indicative of a reduced amount of the miRNA biomarker and that the subject has the disease.

16. The method of detecting a disease of claim 11, wherein two or more miRNA biomarkers validated by the method of any one of claims 1 to 10 are used to determine whether the subject has the disease,
optionally, wherein the two or more miRNA biomarkers are selected from the group consisting of miR-15b, miR-21, miR-375, miR-141, and let-7b.

17. The method of detecting a disease of claim 16, wherein the method determines that the subject has cancer;
optionally, wherein the method determines that the subject has a specific type of cancer.

18. A method of detecting a disease in a subject,
(i) wherein the method comprises incubating a predetermined threshold amount of an osmylated-probe that binds to a miRNA biomarker of the disease with a sample of a specified amount of RNA isolated from the subject, and using a nanopore detection method to determine whether there is an excess of probe in the incubated sample (detection) or an excess of miRNA biomarker in the incubated sample (silencing), wherein silencing is indicative of an elevated amount of the miRNA biomarker and that the subject has the disease, or
(ii) wherein the method comprises incubating a predetermined threshold amount of an osmylated-probe that binds to a miRNA biomarker of the disease with a sample of a specified amount of RNA isolated from the subject, and using a nanopore detection method to determine whether there is an excess of probe in the incubated sample (detection) or an excess of miRNA biomarker in the incubated sample (silencing), wherein detection is indicative of a reduced amount of the miRNA biomarker and that the subject has the disease.

19-20. (canceled)


SEQUENCE LISTING (7 sequences)

SEQ ID NO: 1 - DNA, 20 bp, synthetic construct
  source 1..20: mol_type = other DNA
tctctctctctctctctctc

SEQ ID NO: 2 - DNA, 31 bp, synthetic construct
  source 1..31: mol_type = other DNA
  misc_feature 1..28: note = DNA
  misc_feature 29..30: note = RNA
  misc_feature 31: note = DNA
agagagagccagagagagccagagagccttc

SEQ ID NO: 3 - DNA, 39 bp, synthetic construct
  source 1..39: mol_type = other DNA
  modified_base 6: mod_base = OTHER; note = Uracil
aaaaatcacgcgagccgaacgaacaaactttttcaaaaa

SEQ ID NO: 4 - DNA, 39 bp, synthetic construct
  source 1..39: mol_type = other DNA
  modified_base 6: mod_base = OTHER; note = 2'-O-methyl uracil
  modified_base 12: mod_base = OTHER; note = 2'-O-methyl uracil
  modified_base 16: mod_base = OTHER; note = 2'-O-methyl uracil
  modified_base 18: mod_base = OTHER; note = 2'-O-methyl uracil
  modified_base 21: mod_base = OTHER; note = 2'-O-methyl uracil
  modified_base 26: mod_base = OTHER; note = 2'-O-methyl uracil
  misc_feature 1..5: note = DNA
  misc_feature 6: note = RNA
  misc_feature 7..11: note = DNA
  misc_feature 12: note = RNA
  misc_feature 13..15: note = DNA
  misc_feature 16: note = RNA
  misc_feature 17: note = DNA
  misc_feature 18: note = RNA
  misc_feature 19..20: note = DNA
  misc_feature 21: note = RNA
  misc_feature 22..25: note = DNA
  misc_feature 26: note = RNA
  misc_feature 27..39: note = DNA
aaaaatcaacatcagtctgataagctatttttcaaaaaa

SEQ ID NO: 5 - DNA, 36 bp, synthetic construct
  source 1..36: mol_type = other DNA
  modified_base 8: mod_base = OTHER; note = 2'-O-methyl uracil
  modified_base 10..12: mod_base = OTHER; note = 2'-O-methyl uracil
  modified_base 22: mod_base = OTHER; note = 2'-O-methyl uracil
  modified_base 24..25: mod_base = OTHER; note = 2'-O-methyl uracil
  misc_feature 1..7: note = DNA
  misc_feature 8: note = RNA
  misc_feature 9: note = DNA
  misc_feature 10..12: note = RNA
  misc_feature 13..21: note = DNA
  misc_feature 22: note = RNA
  misc_feature 23: note = DNA
  misc_feature 24..25: note = RNA
  misc_feature 26..36: note = DNA
aaaaccatctttaccagacagtgttatttttaaaaa

SEQ ID NO: 6 - DNA, 39 bp, synthetic construct
  source 1..39: mol_type = other DNA
  modified_base 7: mod_base = OTHER; note = Uracil
  modified_base 9: mod_base = OTHER; note = Uracil
  modified_base 16: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 27: mod_base = OTHER; note = Uracil
aaaaaatgtaaaccatgatgtgctgctatttttaaaaaa

SEQ ID NO: 7 - DNA, 36 bp, synthetic construct
  source 1..36: mol_type = other DNA
  modified_base 17: mod_base = OTHER; note = 2'-O-methyl uracil
  modified_base 20: mod_base = OTHER; note = 2'-O-methyl uracil
  modified_base 24: mod_base = OTHER; note = 2'-O-methyl uracil
  misc_feature 1..16: note = DNA
  misc_feature 17: note = RNA
  misc_feature 18..19: note = DNA
  misc_feature 20: note = RNA
  misc_feature 21..23: note = DNA
  misc_feature 24: note = RNA
  misc_feature 25..36: note = DNA
aaaaaaccacacaacctactacctcatttttaaaaa